(19) United States
(12) Patent Application Publication
(10) Pub. No.: US 2023/0042330 A1
(43) Pub. Date: Feb. 9, 2023

(54) A TOOL FOR SELECTING RELEVANT FEATURES IN PRECISION DIAGNOSTICS

(71) Applicant: PRENOSIS, INC., Chicago, IL (US)

(72) Inventors: Ishan Taneja, Chicago, IL (US); Carlos G. Lopez-Espina, Evanston, IL (US); Sihai Dave Zhao, Champaign, IL (US); Ruoqing Zhu, Savoy, IL (US); Bobby Reddy, JR., Chicago, IL (US)

(21) Appl. No.: 17/791,880

(22) PCT Filed: Jan. 12, 2021
(86) PCT No.: PCT/US2021/013142

§ 371 Date: Jul. 8, 2022

Related U.S. Application Data
(60) Provisional application No. 62/959,754, filed on Jan. 10, 2020.

(51) Int. Cl. G16H 50/50

(52) U.S. Cl. CPC G16H 50/50

(57) ABSTRACT

A method for ranking an unmeasured feature for an instance given at least one feature is measured is provided. The method includes imputing a first value to the unmeasured feature in the instance while holding the other remaining unmeasured features constant and evaluating a first outcome with a model using the first value in the instance. The method includes imputing a second value to the unmeasured feature in the dataset while holding the other remaining unmeasured features constant, evaluating a second outcome with the model using the second value in the instance, and determining a statistical parameter with the first outcome and the second outcome. The method also includes assigning the unmeasured feature a ranking corresponding to the determined statistical parameter. A system and a non-transitory, computer readable medium storing instructions to perform the above method are also presented.

### CROSS REFERENCE TO RELATED APPLICATIONS

The present application claims priority to and the benefit of the U.S. Provisional Patent Application No. 62/959,754 filed Jan. 10, 2020, titled “Tool for Selecting Relevant Features in Precision Diagnostics,” which is hereby incorporated by reference in its entirety as if fully set forth below and for all applicable purposes.

### FIELD OF THE DISCLOSURE

The present disclosure generally relates to selecting data and collection methods and instruments to provide accurate and timely outcome predictions. More specifically, the present disclosure relates to methods and systems to provide educated suggestions to optimize cost and time for data collection with an enhanced confidence level on an individual basis.

### INTRODUCTION

Diagnostic systems based on machine learning (ML) algorithms provide population wide ranking of clinical features in terms of importance. However, when a set of features are collected for a specific patient, the population-wide ranking of the features may not be optimal for the patient. The consequences of collecting less than optimal features to measure may have undesirable outcomes for the patient, especially in emergency situations. It is desirable to have systems and methods that allow the selection of optimal features for completing a predictive dataset on a patient-specific basis.

### SUMMARY

In some embodiments of the present disclosure, a method for ranking an unmeasured feature for an instance given at least one feature is measured, includes imputing a first value to the unmeasured feature in the instance while holding the other remaining unmeasured features constant and evaluating a first outcome with a model using the first value in the instance. The method includes imputing a second value to the unmeasured feature in the dataset while holding the other remaining unmeasured features constant, evaluating a second outcome with the model using the second value in the instance, and determining a statistical parameter with the first outcome and the second outcome. The method also includes assigning the unmeasured feature a ranking corresponding to the determined statistical parameter.

In some embodiments, a system for ranking an unmeasured feature for an instance given at least one feature is measured includes a memory, storing instructions and one or more processors communicatively coupled with the memory. The one or more processors are configured to execute the instructions to cause the system to impute a first value to the unmeasured feature in the instance while holding another remaining unmeasured features constant, and to evaluate a first outcome with a model using the first value in the instance. The one or more processors are also configured to impute a second value to the unmeasured feature in the instance while holding another remaining unmeasured features constant, to evaluate a second outcome with the model using the second value in the instance, and to determine a statistical parameter with the first outcome and the second outcome. The one or more processors are also configured to assign the unmeasured feature a ranking corresponding to the statistical parameter, and to select a filtered dataset from a master dataset according to at least one measured feature from the instance, the master dataset comprising multiple datasets associated with multiple known outcomes.

In some embodiments, a non-transitory, computer readable medium stores instructions which, when executed by a computer, cause the computer to perform a method for ranking an unmeasured feature for an instance given at least one feature is measured. The method includes imputing a first value to the unmeasured feature in the instance while holding another remaining unmeasured features constant and evaluating a first outcome with a model using the first value in the instance. The method also includes imputing a second value to the unmeasured feature in the instance while holding another remaining unmeasured features constant, evaluating a second outcome with the model using the second value in the instance, and determining a statistical parameter with the first outcome and the second outcome. The method also includes assigning the unmeasured feature a ranking corresponding to the statistical parameter, and selecting a filtered dataset from a master dataset according to at least one measured feature from the instance, the master dataset comprising multiple datasets associated with multiple known outcomes. In the method, assigning the unmeasured feature a ranking corresponding to the statistical parameter includes identifying, in a filtered dataset, a relative importance of the unmeasured feature with one or more known outcomes using model-based feature importance methodologies.

In some embodiments, a method for ranking an unmeasured feature for an instance given at least one feature is measured includes selecting a filtered dataset from a master dataset according to at least one measured feature from the instance, the master dataset comprising multiple datasets associated with multiple known outcomes. The method also includes identifying, in the filtered dataset, the relative importance of the unmeasured feature with one or more known outcomes using model-based feature importance methodologies, and assigning the unmeasured feature a ranking corresponding to the output from the model-based feature importance.

In some embodiments, a method for ranking an unmeasured feature for an instance given at least one feature is measured includes accessing a master dataset, the master dataset comprising multiple datasets associated with known outcomes. The method also includes determining a variance value associated with a model for an outcome, the model based on the unmeasured feature and at least one other distinct feature in the dataset, evaluating a variation of prediction for an outcome with the model using multiple imputed values for the unmeasured feature in the dataset; and assigning the unmeasured feature a ranking according to a value of the variation of prediction relative to the variance value.

In some embodiments, a method for ranking an unmeasured feature for an instance given at least one feature is measured, includes determining a rule for assessing a decision value based on a dataset. The dataset includes collected values for multiple measured features in the instance and the unmeasured feature in the instance, and wherein the rule is consistent with: (1) multiple known outcomes from a master dataset that comprises multiple datasets and (2) one or more measured features. The method also includes determining an accuracy of the rule based on the multiple outcome values and the known outcomes for each of the datasets, and assigning the unmeasured feature a ranking corresponding the accuracy of the rule.

In some embodiments, a method to determine a sampling frequency for a selected feature based on a predictability of the feature includes identifying a set of observed features and a set of missing features. The method also includes building a model to predict a sample frequency of a selected feature using a feature matrix selected from a historical dataset, generating a prediction for the sampling frequency using the model, and determining a variance of the selected feature from multiple time predictions. The method also includes ranking the selected feature with respect to other features based on the variance, and increasing the sampling frequency of the selected feature when the rank of the feature is in a pre-determined top percentile.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

The accompanying drawings, which are included to provide further understanding and are incorporated in and constitute a part of this specification, illustrate disclosed embodiments and together with the description serve to explain the principles of the disclosed embodiments. In the drawings:

FIG. 1 illustrates an example architecture suitable for a diagnostic engine in a streaming data environment, in accordance with various embodiments.

FIG. 2 is a block diagram illustrating an example server and client from the architecture of FIG. 1, according to certain aspects of the disclosure.

FIG. 3 illustrates an example workflow for a decision tree, in accordance with various embodiments.

FIG. 4 illustrates a method for ranking one or more features in a dataset according to relevance for a diagnostic engine using a constraint function, in accordance to various embodiments.

FIG. 5 is a block diagram illustrating a method for quantifying the effect of a missing feature in the uncertainty of prediction for a diagnostic engine, in accordance to various embodiments.

FIG. 6 is a block diagram illustrating a method for quantifying the relevance of a feature in a diagnostic engine selecting similar patient datasets from a master dataset, in accordance with various embodiments.

FIG. 7 is a block diagram illustrating a method for quantifying the relevance of a feature in a diagnostic engine using a historical dataset selected from a master dataset, in accordance with various embodiments.

FIG. 8 is a flow chart illustrating steps in a method to select a relevant feature for a diagnostic engine based on multiple medical features received or imputed over a time sequence, in accordance with various embodiments.

FIG. 9 is a flow chart illustrating steps in a method to select a relevant feature for a diagnostic engine by quantifying the effect of missing an individual feature, in accordance with various embodiments.

FIG. 10 is a flow chart illustrating steps in a method to select a relevant feature for a diagnostic engine based on a filter for similar patient population from a master dataset, in accordance with various embodiments.

FIG. 11 is a flow chart illustrating steps in a method to select a relevant feature for a diagnostic engine based on a model for measured features, in accordance to various embodiments.

FIG. 12 is a flow chart illustrating steps in a method to select a relevant feature for a diagnostic engine based on a historical dataset selected from a master dataset, in accordance to various embodiments.

FIG. 13 is a flow chart illustrating steps in a method to build a multivariable model that predicts the importance of missing features using measured features, in accordance to various embodiments.

FIG. 14 is a flow chart illustrating steps in a method to determine a sampling frequency for a selected feature based on a predictability of the feature, in accordance to various embodiments.

FIG. 15 is a block diagram illustrating an example computer system with which the client and server of FIGS. 1 and 2, and the methods of FIGS. 8-12 can be implemented, in accordance with various embodiments.

In the figures, elements and steps denoted by the same or similar reference numerals are associated with the same or similar elements and steps, unless indicated otherwise.

### DETAILED DESCRIPTION

In the following detailed description, numerous specific details are set forth to provide a full understanding of the present disclosure. It will be apparent, however, to one ordinarily skilled in the art, that the embodiments of the present disclosure may be practiced without some of these specific details. In other instances, well-known structures and techniques have not been shown in detail so as not to obscure the disclosure.

### General Overview

Recently, the number of features that can be measured for a patient has increased dramatically. In various embodiments, feature measurements may include: Genomics; Transcriptomics; Proteomics; Metabolomics; Wearable device data; Behavioral data (food/drink purchases, fitness data, and the like); Billing data (insurance, and the like); and Social media data. Different features may have a different cost and acquisition time associated with them. Thus, it is desirable to have a personalized ranking of features.

Accordingly, it is desirable to understand the relevance of a specific feature in the diagnostic of a given patient to reduce the cost and time of measurement, which may be crucial in urgent care situations. The relevance of any given feature may also depend on circumstance and even the patient itself. For instance, a 70-year old patient with a fever, leukocytosis, and history of type 2 diabetes may most benefit from the subsequent measurement of features a, b, and c. On the other hand, an otherwise healthy 23-year old presenting with symptoms of a persistent headache may most benefit from the subsequent measurements of features x, y, and z. Accordingly, it is highly desirable to tailor the ranking of relevant features for a single patient, using data collected from a broad population of individuals.

Given a patient's available information and quantifiable health state, methods and systems as disclosed herein determine valuable features to collect for a corresponding clinical inquiry (e.g., whether the patient has disease d, or will the patient benefit from treatment t, and the like). In addition, various embodiments also determine a frequency of collection, and which measurement technologies may be used to acquire the selected features with a desirable accuracy and precision. Various embodiments provide the optimal set of features that a clinician may collect constrained by the available resources and time to diagnostic when a given patient has had their vitals measured (e.g., current available information). In various embodiments, the feature selection mechanism is conditional on the patient's available information and quantifiable health state.

In accordance to various embodiments, the noise tolerance for a set of features can be determined empirically. In addition, various embodiments may determine the noise tolerance of a feature conditional on a set of features already measured. Accordingly, various embodiments include suggesting to the end user to increase or decrease the average allowable tolerance for a given feature based on previous measurements of that feature, or of other features.

In accordance to various embodiments, the optimal sampling frequency for a set of features can be determined algorithmically. In addition, various embodiments may determine the sampling frequency of a feature conditional on a set of features already measured. Accordingly, various embodiments may include suggesting to the end user to increase or decrease the sampling frequency for a given feature based on previous measurements of that feature, or of other features.

In various embodiments, machine learning algorithms are used to rank feature relevance according to the quantifiable information available for a given patient and a model trained on a dataset consisting of an input feature matrix and an outcome vector. In addition, embodiments consistent with this disclosure provide a subject-specific estimate of the ranking of a set of features based on the quantifiable information available for a given patient and a dataset.

The proposed solution further provides improvements to the functioning of the computer itself because it saves data storage space and reduces network usage due to the shortened time-to-decision resulting from methods and systems as disclosed herein.

Although many examples provided herein describe a patient's data being identifiable, or download history for images being stored, each user may grant explicit permission for such patient information to be shared or stored. The explicit permission may be granted using privacy controls integrated into the disclosed system. Each user may be provided notice that such patient information can or will be shared with explicit consent, and each patient may at any time end having the information shared, and may delete any stored user information. The stored patient information may be encrypted to protect patient security.

### Example System Architecture

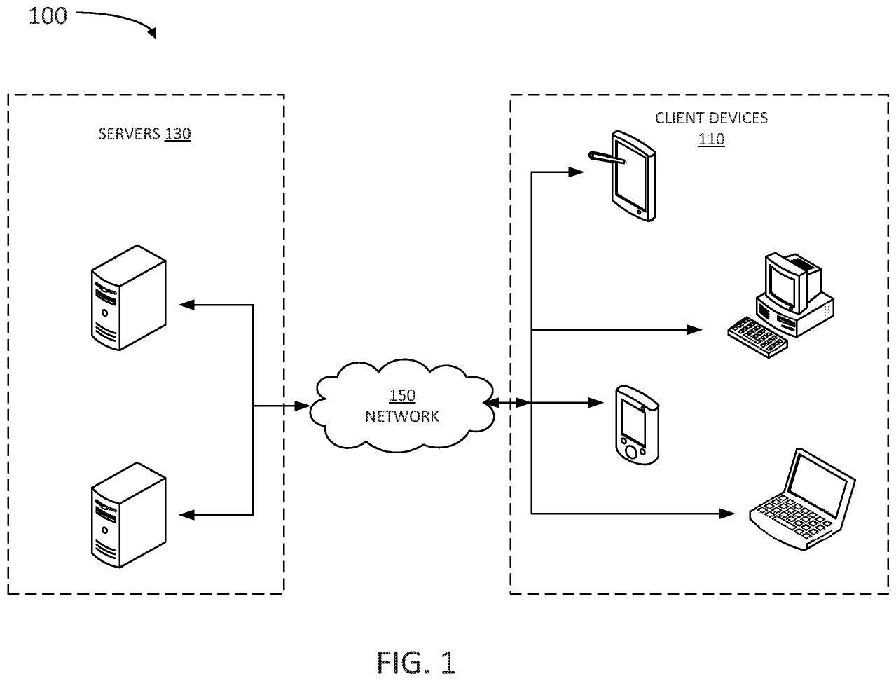

FIG. 1 illustrates an example architecture 100 for a diagnostic engine in a streaming data environment, in accordance with various embodiments. Architecture 100 includes servers 130 and client devices 110 connected over a network 150. One of the many servers 130 is configured to host a memory including instructions which, when executed by a processor, cause the server 130 to perform at least some of the steps in methods as disclosed herein. At least one of servers 130 may include, or have access to, a database including clinical data for multiple patients.

Servers 130 may include any device having an appropriate processor, memory, and communications capability for hosting the collection of images and a trigger logic engine. The trigger logic engine may be accessible by various client devices 110 over network 150. Client devices 110 can be, for example, desktop computers, mobile computers, tablet computers (e.g., including e-book readers), mobile devices (e.g., a smartphone or PDA), or any other devices having appropriate processor, memory, and communications capabilities for accessing the trigger logic engine on one of servers 130. In accordance to various embodiments, client devices 110 may be used by healthcare personnel such as physicians, nurses, or paramedics, accessing the trigger logic engine on one of servers 130 in a real-time emergency situation (e.g., in a hospital, clinic, ambulance, or any other public or residential environment). In some embodiments, one or more users of client devices 110 (e.g., nurses, paramedics, physicians, and other healthcare personnel) may provide clinical data to the trigger logic engine in one or more server 130, via network 150. In yet other embodiments, one or more client devices 110 may provide the clinical data to server 130 automatically. For example, in some embodiments, client device 110 may be a blood testing unit in a clinic, configured to provide patient results to server 130 automatically, through a network connection. Network 150 can include, for example, any one or more of a local area network (LAN), a wide area network (WAN), the Internet, and the like. Further, network 150 can include, but is not limited to, any one or more of the following network topologies, including a bus network, a star network, a ring network, a mesh network, a star-bus network, tree or hierarchical network, and the like.

### Example Diagnostic System

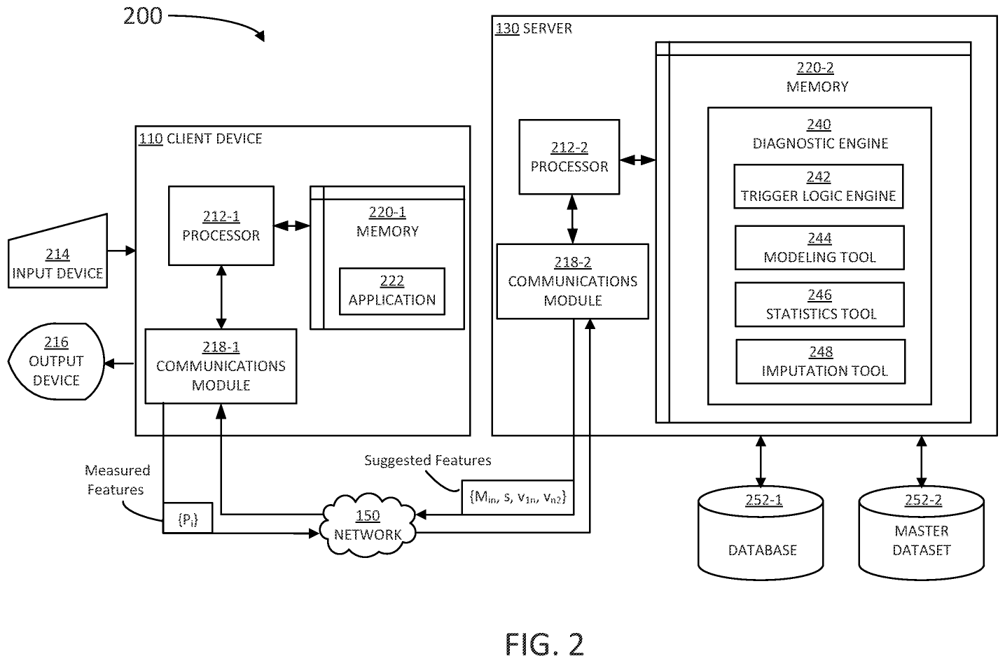

FIG. 2 is a block diagram 200 illustrating an example server 130 and client device 110 in the architecture 100 of FIG. 1, according to certain aspects of the disclosure. Client device 110 and server 130 are communicatively coupled over network 150 via respective communications modules 218-1 and 218-2 (hereinafter, collectively referred to as “communications modules 218”). Communications modules 218 are configured to interface with network 150 to send and receive information, such as data, requests, responses, and commands to other devices on the network. Communications modules 218 can be, for example, modems or Ethernet cards. Client device 110 and server 130 may include a memory 220-1 and 220-2 (hereinafter, collectively referred to as “memories 220”), and a processor 212-1 and 212-2 (hereinafter, collectively referred to as “processors 212”), respectively. Memories 220 may store instructions which, when executed by processors 212 cause either one of client device 110 or server 130 to perform one or more steps in methods as disclosed herein. Accordingly, processors 212 may be configured to execute instructions, such as instructions physically coded into processors 212, instructions received from software in memories 220, or a combination of both.

In accordance with various embodiments, server 130 may include, or be communicatively coupled to, a database 252-1 and a master dataset 252-2 (hereinafter, collectively referred to as “databases 252”). In one or more implementations, databases 252 may store clinical data for multiple patients. Databases 252 may include a historical dataset, H, having time-series measurements for various features, treatment information, model predictions, and outcome information per patient, for one or more patients. Historical database, H, may include multiple features measured at different time points.

In accordance to various embodiments, master dataset 252-2 may be the same as database 252-1, or may be included therein. The clinical data in databases 252 may include metrology information such as non-identifying patient characteristics; vital signs; blood measurements such as complete blood count (CBC), comprehensive metabolic panel (CMP) and blood gas (e.g., Oxygen, CO₂, and the like); immunologic information; biomarkers; culture; and the like. The non-identifying patient characteristics may include age, gender, and general medical history, such as a chronic condition (e.g., diabetes, allergies, and the like). In various embodiments, the clinical data may also include actions taken by healthcare personnel in response to metrology information, such as therapeutic measures, medication administration events, dosages, and the like. In various embodiments, the clinical data may also include events and outcomes occurring in the patient's history (e.g., sepsis, stroke, cardiac arrest, shock, and the like). Although databases 252 are illustrated as separated from server 130, in certain aspects databases 252 and trigger logic engine 242 can be hosted in the same server 130, and be accessible by any other server or client device in network 150.

Memory 220-2 in server 130 may include a diagnostic engine 240, for evaluating a likely patient outcome based on a dataset of medical features. Diagnostic engine 240 may also include a trigger logic engine 242, a modeling tool 244, a statistics tool 246, and an imputation tool 248. Modeling tool 244 may include instructions and commands to collect relevant clinical data and evaluate a probable outcome (e.g., a diagnostic). In some embodiments, modeling tool 244 may suggest an action to take from a plurality of possible actions. Modeling tool 244 may include commands and instructions from a neural network (NN), such as a deep neural network (DNN), a convolutional neural network (CNN), a generative adversarial neural network (GAN), a deep reinforcement learning (DRL) algorithm, a deep recurrent neural network (DRNN), a classic machine learning algorithm such as random forest, k-nearest neighbor (KNN) algorithm, k-means clustering algorithms, or any combination thereof. According to various embodiments, modeling tool 244 may include a machine learning algorithm, an artificial intelligence algorithm, or any combination thereof. Modeling tool 244 may dynamically generate models based model based on predictions made at certain time points, measurements made for a set of patients, and actual outcomes for a set of patients, with information extracted from historical dataset, H.

Statistics tool 246 evaluates data stored in databases 252, or provided by modeling tool 244. Imputation tool 248 may provide modeling tool 244 with data inputs otherwise missing from metrology information collected by trigger logic engine 242. Trigger logic engine 242 may be configured to evaluate various metrics associated with input data {Pᵢ} and model F computed by the statistics tool, and trigger an action based on the input and whether it satisfies a certain condition. The streaming data input {Pᵢ} may include multiple measured features provided by a nurse or other medical personnel using client device 110 for a patient i. In accordance to some embodiments, server 130 may provide a ranking variable for one or more features in {Mᵢ} to client device 110. The ranking variable provided for a given feature in {Mᵢ} may be information used by the end-user to determine which features or set of features to subsequently measure for a given patient. In accordance with some embodiments, measured features {Pᵢ} are provided to server 130 from one or more client devices 110. In accordance with various embodiments, client device 110 may receive, in response to input data {Pᵢ}, a predicted outcome or diagnostic from server 130.

Modeling tool 244 includes a model F trained on a dataset D consisting of an m×(l+k) dimensional input feature matrix X and an outcome vector Y of dimension m (one entry for each patient). Mᵢ is a k-dimensional feature vector including k features not measured for subject i. An i-dimensional feature vector Pᵢ for subject i includes l features measured for subject i. Accordingly, a set of n missing features (Mᵢₙ, wherein n≤k) may be selected from Mᵢ. For a given Mᵢₙ, diagnostic engine 240 assigns three values. A first value is a scalar value, s (e.g., 0≤s≤1), indicative of the importance of the n-set with respect to Y (the patient outcome). A second value is a vector of size n (vᵢₙ), wherein each entry corresponds to a time-dependent variation for each feature in a given n-set. And a third value, another vector of size n, v₂ₙ, is indicative of the maximum allowable noise in the measurement of each missing feature in the n-set. According to various embodiments, server 130 transmits the group {Mᵢₙ, s, v₁ₙ, v₂ₙ} to client device 110.

Client device 110 may access diagnostic engine 240 through an application 222 or a web browser installed in client device 110. Processor 212-1 may control the execution of application 222 in client device 110. In accordance to various embodiments, application 222 may include a user interface displayed for the user in an output device 216 of client device 110 (e.g., a graphical user interface—GUI—). A user of client device 110 may use an input device 214 to enter input data as a metrology information or submit a query to diagnostic engine 240 via the user interface of application 222. Input device 214 may include a stylus, a mouse, a keyboard, a touch screen, a microphone, or any combination thereof. Output device 216 may also include a display, a headset, a speaker, an alarm or a siren, or any combination thereof.

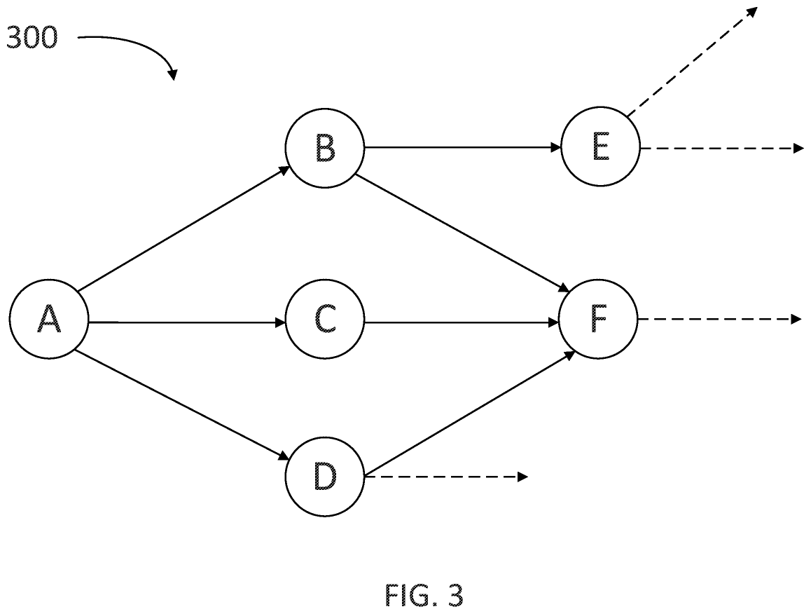

FIG. 3 illustrates an example workflow for a decision tree 300, in accordance with various embodiments. In various embodiments, one or more client devices and servers as disclosed herein may intervene for decision making in each node of decision tree 300. More specifically, in one or more of the nodes in decision tree 300, a diagnostic engine including a trigger logic engine, a modeling tool, a statistics tool, and an imputation tool may be used. Each decision point is independently resolved, potentially leading to a follow up decision. Decisions subsequent to a first decision (A) can commence by using data collected up to the previous decisions, instead of recommend new data. In one exemplary embodiment, a first decision point may include finding patients with high risk of developing sepsis in the next X hrs. A second decision point may include, for those patients, selecting a sub-type of host response benefiting from broad spectrum (A, B, X, and the like).

In various embodiments, a two-layer deep decision tree can be outlined as follows: 1) Clinician inquires if their patient has a high risk of developing sepsis within the next 6 hours. 2a) When the clinician evaluates that the patient is at high risk after receiving relevant information (vitals, labs, machine-learning based predictions), should the patient be given antibiotics or antivirals? 2b). When the clinician thinks the patient does not have sepsis, the next level includes identifying whether the patient has a urinary tract infection (uncomplicated).

Each decision point may include executing a specific workflow. After a root level decision point, subsequent decision points may recommend a set of features to collect. In various embodiments, a diagnostic tool may include several options to recommend a set of features based on a population-wide estimate or to use what is available in record thus far, with data collected according to tests requested or executed at prior decision points (e.g., from historical dataset, H).

In various embodiments, the recommended action may include collecting a new observation for a given patient and move to the next step whenever any new data is ready, regardless of whether all features are available.

In various embodiments, a machine learning model in the modeling tool provides an outcome prediction or an outcome probability, and a confidence level for the prediction, based on available data. In some embodiments, the confidence level may be provided by a statistics tool in the diagnostic engine. Accordingly, there may be one or more decisions available, based on the outcome prediction. A rule, dependent on the one or more decisions, defined in the trigger logic engine may be used to decide when the diagnostics engine is ready to provide an answer or request an action.

The statistics tool may also assess a risk for each of the one or more decisions. When the risk is low, the workflow stops and the decision is taken. When the risk is high, the diagnostic engine may issue a query to the physician, nurse, or other medical personal (e.g., a question displayed on a touchscreen or via a microphone, in the client device). When the physician, nurse, or other personnel has a positive response to the query (‘OK’ response, or press a button on the touchscreen of the client device), the workflow stops and the decision is taken.

When the system detects missing data before making a decision (e.g., due to high risk or a low confidence level), the diagnostic engine may decide to wait for at least one or more features in the missing data to be measured and incorporated in the modeling tool. Based on a selected decision, the system may suggest the user to collect a new set of features. In accordance to various embodiments, the system may wait for new features to be collected, even when not requested by a user. In accordance to various embodiments, the modeling tool may also update the model based on available features with associated confidence metric.

According to various embodiments, to suggest features for collection in the case of insufficient confidence on the predicted outcome, the system may quantify the effect of missing an individual feature on the uncertainty of prediction. In various embodiments, to suggest features to be collected, the system may also apply a dynamic model and variable importance determination based on ‘similar’ patient population. A variable importance prediction may be obtained based on available variables. To suggest a missing feature, the system may also quantify the added predictive value of the feature using the historical dataset, H.

In various embodiments, the diagnostic engine also provides a ranking variable assigned to each feature or set of features. Accordingly, the diagnostic engine may suggest the missing features to be measured based on their rank and a user-specified constraint function. The constraint function may include a cost of the feature and an acquisition time.

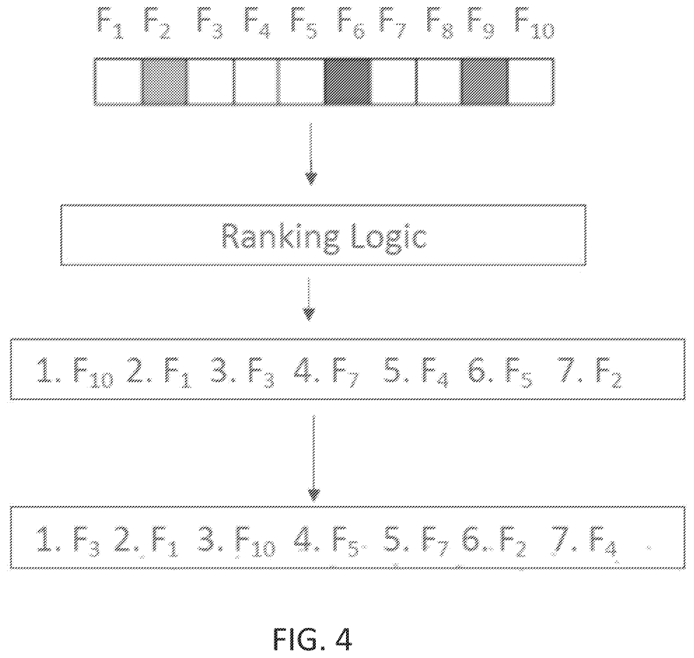

FIG. 4 illustrates a method for ranking one or more features in a dataset, according to relevance for a diagnostic engine using a constraint function, in accordance to various embodiments. Assuming that there may be up to ten features to obtain, F₁, F₂, F₃, F₄, F₅, F₆, F₇, F₈, F₉, and F₁₀, wherein three of the features are measured {P=F₂, F₆, F₉}, for a specific patient. For example, the patient may enter an emergency room in a hospital and at least two of features F₂, F₆, and F₉ may include temperature and heart rate. The table below lists the cost and time to obtain (delay) involved with measurement of the missing features {M=F₁, F₃, F₄, F₅, F₇, F₈, and F₁₀}.

| Cost | A$ | B$ | A$ | C$ | C$ | B$ | B$ |
|---|---|---|---|---|---|---|---|
| Time | 1 h | 2 h | 1 h | 3 h | 3 h | 2 h | 2 h |

Based on the available data {P}, a clinician may inquire whether the patient has disease d. Accordingly, a diagnostic tool as disclosed herein outputs a ranking for the relevance of the remaining features {M}, in terms of predicting the patient outcome with a high confidence level. A decision may be time sensitive (e.g., within the next hour or other prescribed amount of time) and cost may be a secondary concern. Accordingly, a diagnostic engine may include a constraint function (e.g., ranking logic) in the modeling tool that reflects the above configuration with a factor proportional to a mathematical expression as follows:

$$\frac{1}{importance * \sqrt{cost} * {time}^{2}} . \qquad ( 1 )$$

Features in set {M} may be presented to the clinician in descending order according to the value of the constraint function. In some embodiments, of the N features presented (n=7 in this example), the diagnostic tool may suggest measurement of the top \sqrt{N} features in the list (e.g., 3 features: F₂, F₆, and F₉). In various embodiments, this process repeats until the statistics tool in the diagnostic engine reaches a satisfactory value for the confidence level (above a pre-determined threshold).

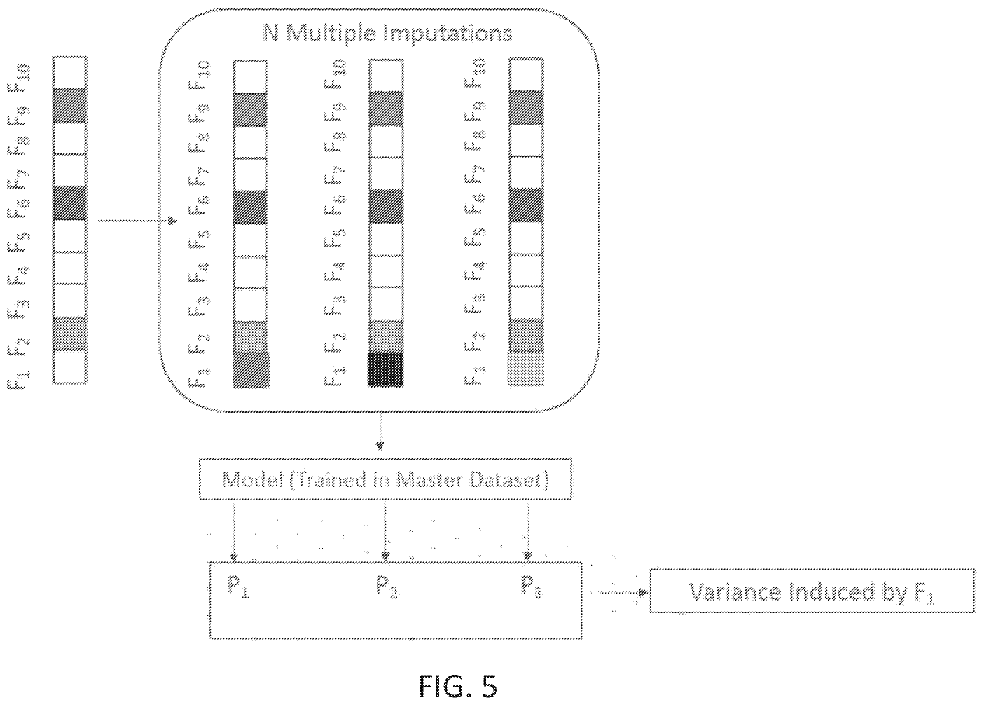

FIG. 5 is a block diagram illustrating a method for quantifying the effect of a missing feature in the uncertainty of prediction for a diagnostic engine, in accordance to various embodiments. In some embodiments, the missing feature set may include an individual feature (n=1 in the n-set, cf. FIG. 2). The uncertainty of prediction is obtained by holding a set of features ‘constant’ (e.g., features F₂, F₆, and F₉, cf FIG. 4) except for the one we are attempting to quantify the prediction uncertainty (e.g., F₁, cf FIG. 4). The modeling tool evaluates a predicted outcome for N-multiple imputations, each of which has a different imputed value for F₁. In some instances, the statistics tool then determines a statistical parameter based on the predictions of the modeling tool. For example, the statistics tool may determine a variance between the N predictions of the modeling tool. In various embodiments, a higher variance found by the statistics tool may be associated with a larger impact on the value of the prediction and hence a larger importance of feature F₁ for the diagnostic of this particular patient.

More specifically, the diagnostic engine quantifies the prediction uncertainty induced by a certain feature in M as follows: hold F₃-F₁₀ ‘constant’, impute F₁ multiple times (N-times) with different values, and calculate the variance in the prediction. Start with a model trained with a fixed number of features (e.g., a large set of features, or a Master Dataset extracted from the historical dataset H, and the like) that produces a diagnostic with a given probability and confidence level.

Assuming that there are j features in P, and there are k features in M for a given patient, the diagnostic engine may perform the following steps:

For i varying from 1 to k:

Select a missing feature Fᵢ from the set M.

Impute features 1 . . . k-minus Fᵢ-in M (e.g., {1 . . . k/i}) with a crude estimate (random, mean, median, and the like) from the historical dataset.

Impute feature Fᵢ via a multiple imputation framework using the historical master dataset, H, generating N imputed values for feature i. Let M_{imputed} refer to an N-vector where each entry corresponds to one of the N imputed values of Fᵢ.

Generate N identical copies of P, M_{{1 . . . k/i}}.

Concatenate P, M_{{1 . . . k/i}}, M_{imputed}, yielding an (N by (j+k)) input matrix I.

Provide, with the modeling tool, a set of N predictions (e.g., diagnostic values or outcomes), one per row in I.

Determine a between imputation variance, bᵢ from the N predictions. After repeating the process for each feature in M, the k values bᵢ may be associated with a relative feature relevance within the set M.

Order each feature in descending order by bᵢ (higher bᵢ corresponds to higher relevance).

In various embodiments, the model used to find the predictions may be fixed, and multiple imputation based on a Master Dataset or historical dataset H is used to rank variable importance. In various embodiments, the model may be dynamically updated as desired.

In various embodiments, the above method may be generalized to ranking separate sets of features by replacing 1 . . . k with a specific list of sets (e.g., [{1, 2, 3}, {1, 3, 4}, {1, 3, 5}, and the like]).

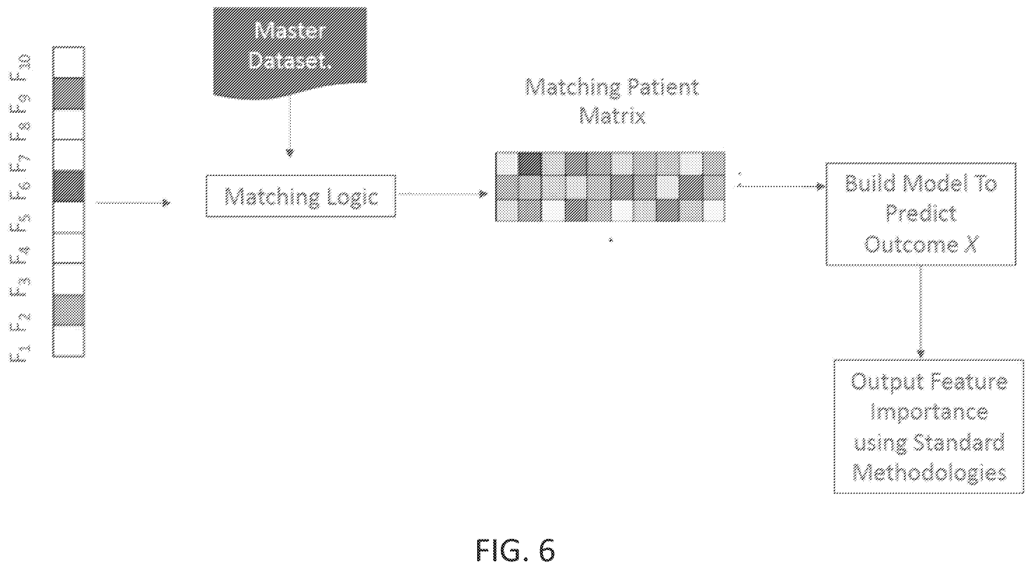

FIG. 6 is a block diagram illustrating a method for quantifying the relevance of a feature in a diagnostic engine selecting similar patient datasets from a master dataset, in accordance with various embodiments. In various embodiments, the method finds a filtered dataset that includes a subset of similar patients to the current patient (e.g., from a master dataset or historical dataset, H). In various embodiments, building a model using a more homogenous population consisting of patients whom ‘look alike’ yields a relevance ranking of features specific to the current patient. The modeling tool builds a new model or updates an existing model to predict the known outcomes for the subset of similar patients (e.g., vector Y) and provides a relevance value or ranking to the missing feature using techniques as disclosed herein.

For a given observation, Pᵢ, of a patient, i, with a limited set of features, the diagnostic engine selects a set, NS, of nearest subjects from the historical master dataset H. The set NS may also include a set X of additional measured features. In some embodiments, the selection of set NS is based on the initial set of limited features. In various embodiments, the set NS can be defined using multiple methods (k-nearest neighbors, fixed radius nearest neighbor, and the like) using any one of different metrics (euclidean, manhattan, mahalanobis, minkowski, chebychev, cosine, correlation, hamming, jaccard, spearman, gaussian kernel, and the like).

In various embodiments, the size of set NS may be an adjustable input in the method. For example, in various embodiments, all subjects may be used. Using the set NS in Master Dataset, and a desirable prediction value (e.g., the known outcomes, Y, for the patients in set NS), the modeling tool builds a supervised model F_{NS} using features X. If the performance of F_{NS} (e.g., outcome prediction and confidence level) is not above a pre-determined threshold (e.g., accuracy, AUC, AUPR, F1-score, sensitivity, specificity, PPV, NPV, RMSE, r², AIC, BIC, and the like), the modeling tool updates the set X and builds a new model F_{NS} (or updates an existing model).

When the model F_{NS} is satisfactory, the modeling tool computes a variable importance of F_{NS}. The variable importance of F_{NS} provides a numerical value for each feature in X via any one of multiple methods. In various embodiments, the variable importance may be provided by model information approaches (such as linear regression, logistic regression, SVM, tree-based methods, neural networks, and the like). Such methods include gini importance, permutation-based importance, coefficient magnitude, and the like. In various embodiments with limited model-specific capabilities, non-model information methods that utilize search algorithms such as Hill Climbing, Simulated Annealing, Genetic-based Algorithms, etc. that optimize for general metrics such as accuracy, AUC, AUPR, F1-score, sensitivity, specificity, PPV, NPV, RMSE, r\hat{ }2, AIC, BIC, and the like. Accordingly, the diagnostic engine suggests new features for patient measurement based on a ranking of the variable importance of the F_{NS}.

In various embodiments, a model F_{NS} may be built or updated for each new feature suggestion. In various embodiments, when a feature is in Mᵢ but not present in X, the feature importance for that feature can or will correspond to NA (not available).

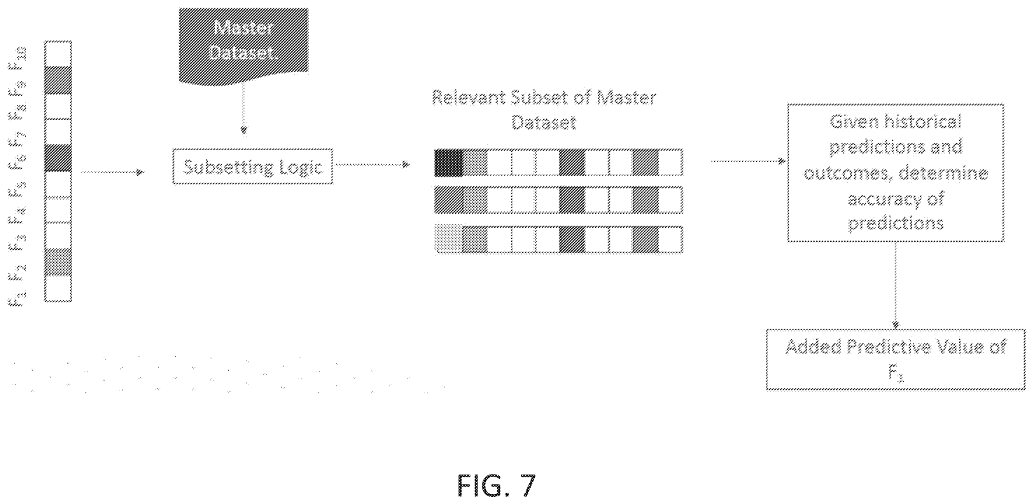

FIG. 7 is a block diagram illustrating a method for quantifying the relevance of a feature in a diagnostic engine using a historical dataset selected from a master dataset, in accordance with various embodiments. Various embodiments use this method to leverage historical dataset H, which includes predictions and corresponding retrospective outcomes. Given a set of present features Pᵢ, and a set of k missing features, Mᵢ, for a patient, j, the diagnostic engine searches through the historical dataset, H, and determines which features, additional to the ones already present in Pᵢ, had the largest impact on predictive accuracy.

The diagnostic engine selects a subset, Hₚ, of H according to instances where only features in Pi are present. In various embodiments, a clinician or any other authorized user may also have the option to subset, or ‘curate’ Hₚ further by selecting a set of nearest subjects to Pi and a distance metric using various methods (k-nearest neighbors, fixed radius nearest neighbor, etc.) with different distance metrics (euclidean, manhattan, mahalanobis, minkowski, chebychev, cosine, correlation, hamming, jaccard, spearman, gaussian kernel, and the like).

For an index j running from 1 . . . k, the method proceeds as follows, in various embodiments: Select a feature Fⱼ in set Mᵢ. Select a subset Hₚ₊ⱼ of H according to instances where features in Pᵢ are present and feature Fⱼ is also present. For Hₚ₊ⱼ, determine the accuracy of model based predictions, Aj, based on known outcomes (Y) using standard metrics like accuracy, AUC, AUPR, F1-score, sensitivity, specificity, PPV, NPV, RMSE, r², AIC, BIC, and the like. Order each feature Fⱼ in M in descending order based on the corresponding values Aⱼ.

In various embodiments, the above method may be generalized to ranking selected sets of n-features by replacing the missing features 1 . . . k with a list of n-sets of missing features in each of the above steps (e.g [{1, 2, 3}, {1, 3, 4}, {1, 3, 5}, and the like]).

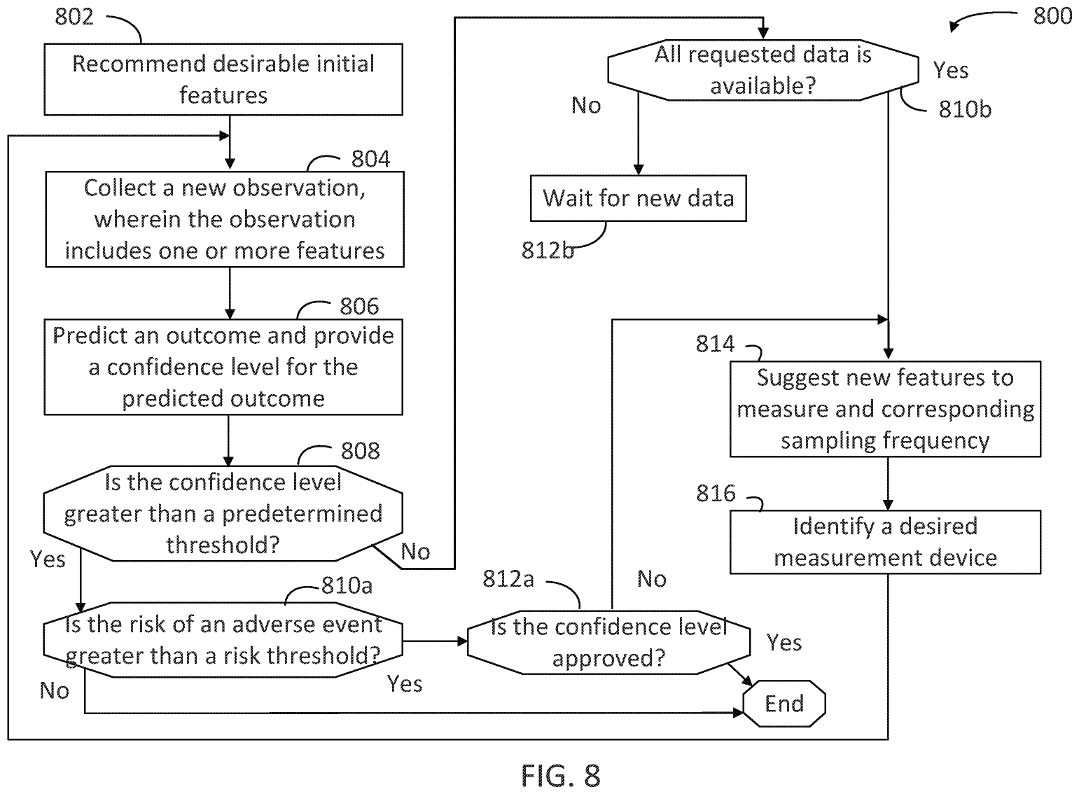

FIG. 8 is a flow chart illustrating steps in a method 800 to perform a medical action on a patient based on multiple medical features received or imputed over a time sequence, in accordance with various embodiments. Method 800 may be performed at least partially by any one of client devices coupled to one or more servers through a network (e.g., any one of servers 130 and any one of client devices 110, and network 150). For example, in accordance to various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 800 may be performed by a computer having a processor executing commands stored in a memory of the computer (e.g., processors 212 and memories 220). In accordance to various embodiments, the user may activate an application in the client device to access, through the network, a diagnostic engine in the server (e.g., application 222 and diagnostic logic engine 240). The diagnostic engine may include a trigger logic engine, a modeling tool, a statistics tool, and an imputation tool to retrieve, supply, and process clinical data in real-time, and provide an action recommendation thereof (e.g., trigger logic engine 242, modeling tool 244, statistics tool 246, and imputation tool 248). Further, steps as disclosed in method 800 may include retrieving, editing, and/or storing files in a database that is part of, or is communicably coupled to, the computer, using, inter-alia, the diagnostic engine (e.g., databases 252). Methods consistent with the present disclosure may include at least some, but not all, of the steps illustrated in method 800, performed in a different sequence. Furthermore, methods consistent with the present disclosure may include at least two or more steps as in method 800 performed overlapping in time, or almost simultaneously.

Step 802 includes recommending a set of desirable initial features to collect. In various embodiments, step 802 includes providing a suggestion based on population-wide estimates or using what is available in record thus far.

Step 804 includes collecting a new observation, wherein the observation includes one or more features. In various embodiments, step 804 may include receiving, from a physician, nurse or other healthcare personnel, a request for one or more features based on feature importance, cost constraints, and time constraints. In various embodiments, step 804 includes collecting one or more new features measured for a given patient. In various embodiments, step 804 includes moving to the next step once any new feature is available. In various embodiments, step 804 includes waiting for a pre-determined set of features to be measured before proceeding.

Step 806 includes predicting an outcome and providing a confidence level for the predicted outcome. In various embodiments, step 806 includes using a machine learning model to provide prediction and/or probability.

Step 808 includes determining whether the confidence level is greater than a pre-determined threshold. In various embodiments, step 808 includes evaluating whether a decision is ready based on a rule dependent on the decision. When the decision is ready, step 808 may include displaying the score and assess the risk of the decision in step 810a. When the risk of an adverse event is lower than a risk threshold in step 810a the workflow ends.

Step 812a includes requesting an approval from a physician, nurse, or healthcare personnel when the risk of an adverse event is higher than the risk threshold. When the healthcare personnel approves the request in step 812a, the workflow ends. When the healthcare personnel does not approve the request in step 812a, step 814 includes providing a ranking variable of importance (s), a sampling frequency (ν₁ₙ) for a given set of unmeasured features.

Step 816 includes identifying a noise tolerance for the given set of unmeasured features (ν₂ₙ). In various embodiments, step 816 includes selecting a measurement technology for each feature based on one or a combination of methods related to noise tolerance consistent with the present disclosure.

When the confidence level is lower than the pre-determined threshold in step 808, step 810b includes determining whether all the requested data is available. If all requested data is not available, the user proceeds to step 812b which entails waiting for new data. When all requested data is available according to step 810b, the method continues in step 814.

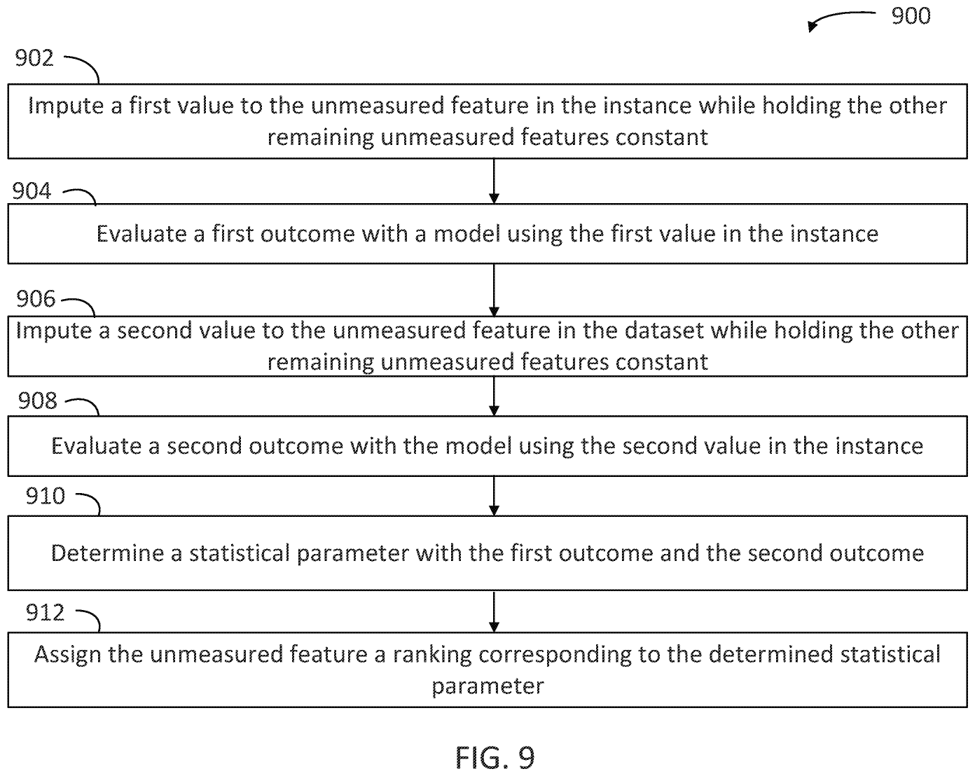

FIG. 9 is a flow chart illustrating steps in a method 900 to select a relevant feature for a diagnostic engine by quantifying the effect of missing an individual feature, in accordance with various embodiments. Method 900 may be performed at least partially by any one of client devices coupled to one or more servers through a network (e.g., any one of servers 130 and any one of client devices 110, and network 150). For example, in accordance to various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 900 may be performed by a computer having a processor executing commands stored in a memory of the computer (e.g., processors 212 and memories 220). In accordance to various embodiments, the user may activate an application in the client device to access, through the network, a diagnostic engine in the server (e.g., application 222 and diagnostic logic engine 240). The diagnostic engine may include a trigger logic engine, a modeling tool, a statistics tool, and an imputation tool to retrieve, supply, and process clinical data in real-time, and provide an action recommendation thereof (e.g., trigger logic engine 242, modeling tool 244, statistics tool 246, and imputation tool 248). Further, steps as disclosed in method 900 may include retrieving, editing, and/or storing files in a database that is part of, or is communicably coupled to, the computer, using, inter-alia, the diagnostic engine (e.g., databases 252). Methods consistent with the present disclosure may include at least some, but not all, of the steps illustrated in method 900, performed in a different sequence. Furthermore, methods consistent with the present disclosure may include at least two or more steps as in method 900 performed overlapping in time, or almost simultaneously.

Step 902 includes imputing a first value to the unmeasured feature in the instance while holding the other remaining unmeasured features constant.

Step 904 includes evaluating a first outcome with a model using the first value in the instance.

Step 906 includes imputing a second value to the unmeasured feature in the dataset while holding the other remaining unmeasured features constant.

Step 908 includes evaluating a second outcome with the model using the second value in the instance.

Step 910 includes determining a statistical parameter with the first outcome and the second outcome.

Step 912 includes assigning the unmeasured feature a ranking corresponding to the determined statistical parameter.

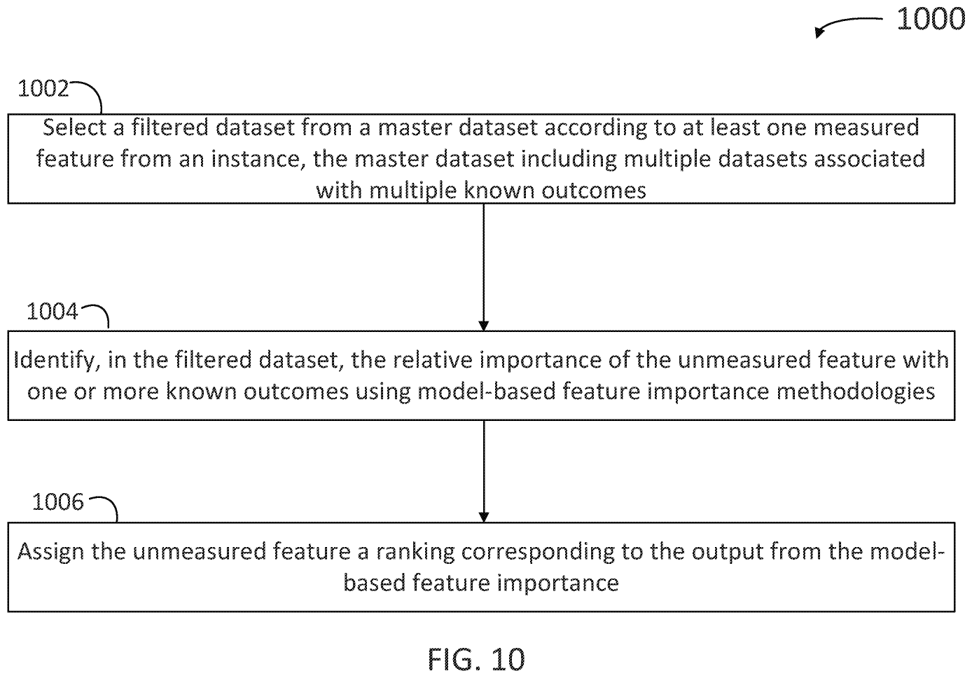

FIG. 10 is a flow chart illustrating steps in a method 1000 to select a relevant feature for a diagnostic engine based on a filter for similar patient population from a master dataset, in accordance with various embodiments. Method 1000 may be performed at least partially by any one of client devices coupled to one or more servers through a network (e.g., any one of servers 130 and any one of client devices 110, and network 150). For example, in accordance to various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 1000 may be performed by a computer having a processor executing commands stored in a memory of the computer (e.g., processors 212 and memories 220). In accordance to various embodiments, the user may activate an application in the client device to access, through the network, a diagnostic engine in the server (e.g., application 222 and diagnostic logic engine 240). The diagnostic engine may include a trigger logic engine, a modeling tool, a statistics tool, and an imputation tool to retrieve, supply, and process clinical data in real-time, and provide an action recommendation thereof (e.g., trigger logic engine 242, modeling tool 244, statistics tool 246, and imputation tool 248). Further, steps as disclosed in method 1000 may include retrieving, editing, and/or storing files in a database that is part of, or is communicably coupled to, the computer, using, inter-alia, the diagnostic engine (e.g., databases 252). Methods consistent with the present disclosure may include at least some, but not all, of the steps illustrated in method 1000, performed in a different sequence. Furthermore, methods consistent with the present disclosure may include at least two or more steps as in method 1000 performed overlapping in time, or almost simultaneously.

Step 1002 includes selecting a filtered dataset from a master dataset according to at least one measured feature from the instance, the master dataset comprising multiple datasets associated with multiple known outcomes.

Step 1004 includes identifying, in the filtered dataset, the relative importance of the unmeasured feature with one or more known outcomes using model-based feature importance methodologies

Step 1006 includes assigning the unmeasured feature a ranking corresponding to the output from the model-based feature importance.

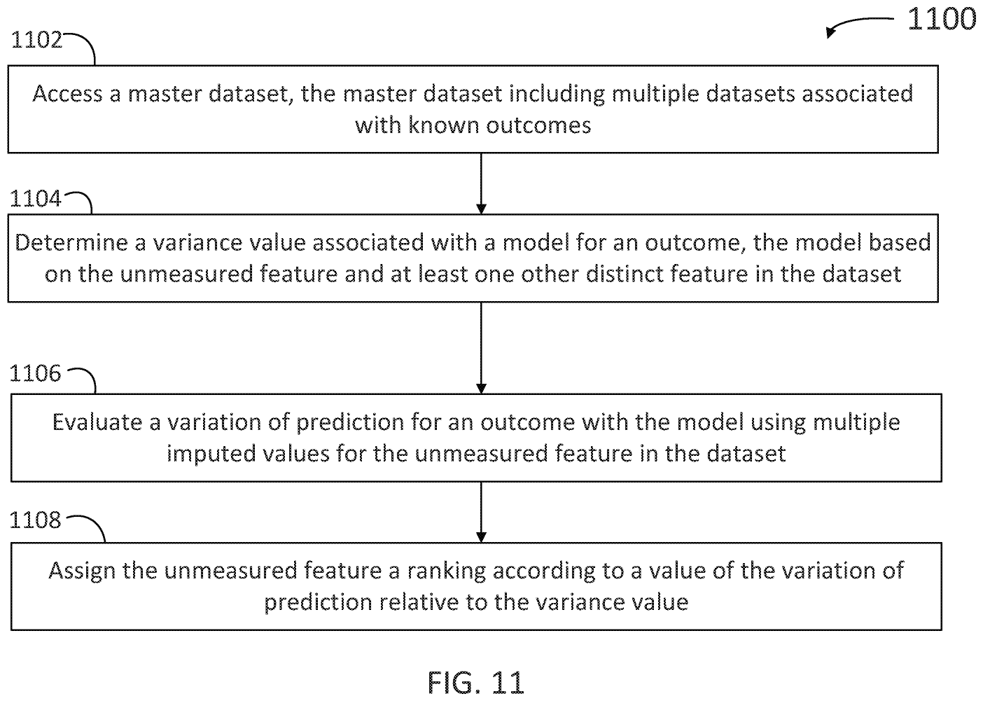

FIG. 11 is a flow chart illustrating steps in a method 1100 to select a relevant feature for a diagnostic engine based on a model for measured features, in accordance to various embodiments. Method 1100 may be performed at least partially by any one of client devices coupled to one or more servers through a network (e.g., any one of servers 130 and any one of client devices 110, and network 150). For example, in accordance to various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 800 may be performed by a computer having a processor executing commands stored in a memory of the computer (e.g., processors 212 and memories 220). In accordance to various embodiments, the user may activate an application in the client device to access, through the network, a diagnostic engine in the server (e.g., application 222 and diagnostic logic engine 240). The diagnostic engine may include a trigger logic engine, a modeling tool, a statistics tool, and an imputation tool to retrieve, supply, and process clinical data in real-time, and provide an action recommendation thereof (e.g., trigger logic engine 242, modeling tool 244, statistics tool 246, and imputation tool 248). Further, steps as disclosed in method 1100 may include retrieving, editing, and/or storing files in a database that is part of, or is communicably coupled to, the computer, using, inter-alia, the diagnostic engine (e.g., databases 252). Methods consistent with the present disclosure may include at least some, but not all, of the steps illustrated in method 1100, performed in a different sequence. Furthermore, methods consistent with the present disclosure may include at least two or more steps as in method 1100 performed overlapping in time, or almost simultaneously.

Step 1102 includes accessing a master dataset, comprising multiple datasets associated with known outcomes.

Step 1104 includes determining a variance value associated with a model for an outcome, the model based on the unmeasured feature and at least one other distinct feature in the dataset.

Step 1106 includes evaluating a variation of prediction for an outcome with the model using multiple imputed values for the unmeasured feature in the dataset.

Step 1108 includes assigning the unmeasured feature a ranking according to a value of the variation of prediction relative to the variance value.

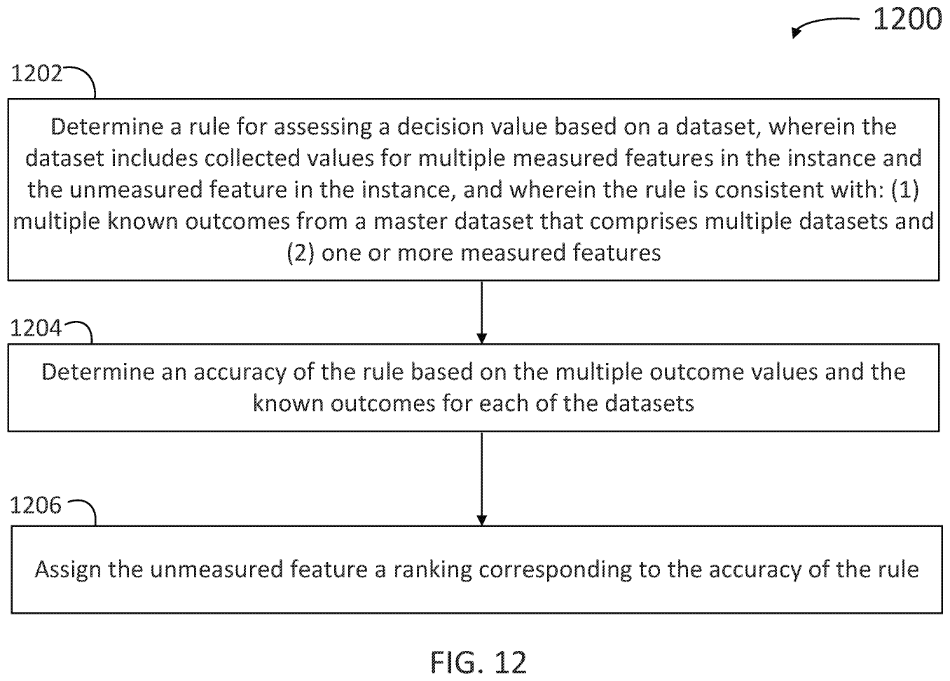

FIG. 12 is a flow chart illustrating steps in a method 1200 to select a relevant feature for a diagnostic engine based on a historical dataset selected from a master dataset, in accordance to various embodiments. Method 1200 may be performed at least partially by any one of client devices coupled to one or more servers through a network (e.g., any one of servers 130 and any one of client devices 110, and network 150). For example, in accordance to various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 1200 may be performed by a computer having a processor executing commands stored in a memory of the computer (e.g., processors 212 and memories 220). In accordance to various embodiments, the user may activate an application in the client device to access, through the network, a diagnostic engine in the server (e.g., application 222 and diagnostic logic engine 240). The diagnostic engine may include a trigger logic engine, a modeling tool, a statistics tool, and an imputation tool to retrieve, supply, and process clinical data in real-time, and provide an action recommendation thereof (e.g., trigger logic engine 242, modeling tool 244, statistics tool 246, and imputation tool 248). Further, steps as disclosed in method 1200 may include retrieving, editing, and/or storing files in a database that is part of, or is communicably coupled to, the computer, using, inter-alia, the diagnostic engine (e.g., databases 252). Methods consistent with the present disclosure may include at least some, but not all, of the steps illustrated in method 1200, performed in a different sequence. Furthermore, methods consistent with the present disclosure may include at least two or more steps as in method 1200 performed overlapping in time, or almost simultaneously.

Step 1202 includes determining a rule for assessing a decision value based on a dataset, wherein the dataset includes collected values for multiple measured features in the instance and the unmeasured feature in the instance, and wherein the rule is consistent with: (1) multiple known outcomes from a master dataset that comprises multiple datasets and (2) one or more measured features.

Step 1204 includes determining an accuracy of the rule based on the multiple outcome values and the known outcomes for each of the datasets.

Step 1206 includes assigning the unmeasured feature a ranking corresponding to the accuracy of the rule.

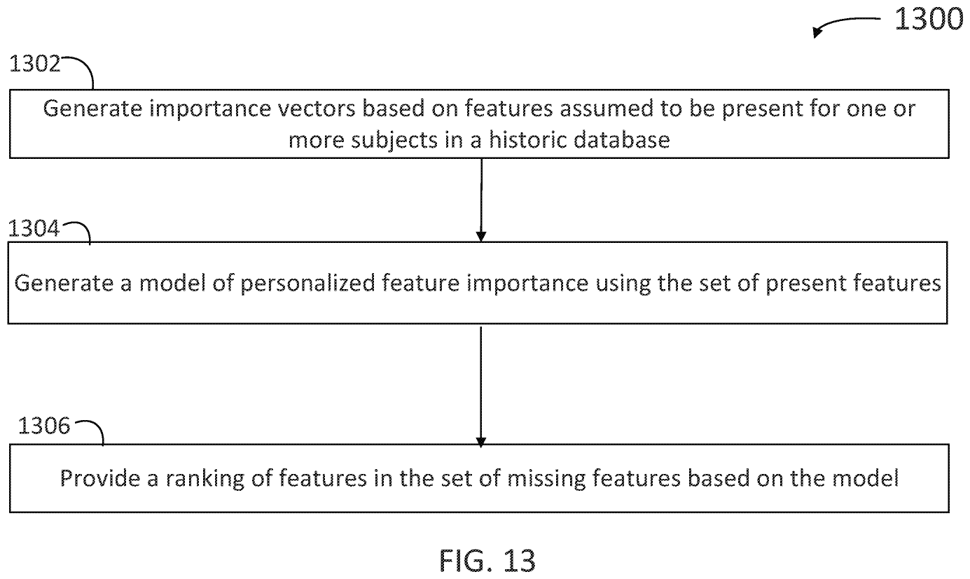

FIG. 13 is a flow chart illustrating steps in a method 1300 to build a multivariable model that predicts the importance of missing features using measured features, in accordance to various embodiments. Method 1300 may be performed at least partially by any one of client devices coupled to one or more servers through a network (e.g., any one of servers 130 and any one of client devices 110, and network 150). For example, in accordance to various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 1200 may be performed by a computer having a processor executing commands stored in a memory of the computer (e.g., processors 212 and memories 220). In accordance to various embodiments, the user may activate an application in the client device to access, through the network, a diagnostic engine in the server (e.g., application 222 and diagnostic logic engine 240). The diagnostic engine may include a trigger logic engine, a modeling tool, a statistics tool, and an imputation tool to retrieve, supply, and process clinical data in real-time, and provide an action recommendation thereof (e.g., trigger logic engine 242, modeling tool 244, statistics tool 246, and imputation tool 248). Further, steps as disclosed in method 1300 may include retrieving, editing, and/or storing files in a database that is part of, or is communicably coupled to, the computer, using, inter-alia, the diagnostic engine (e.g., databases 252). Methods consistent with the present disclosure may include at least some, but not all, of the steps illustrated in method 1300, performed in a different sequence. Furthermore, methods consistent with the present disclosure may include at least two or more steps as in method 1300 performed overlapping in time, or almost simultaneously.

The basic idea behind the third method is to build a multi-class model that predicts the importance of features not measured using features that are available for a given subject. This model is created by generating a dataset that estimates the variance induced by a specific feature in M for all relevant subjects in the historical dataset H for all features in M. The methodology is suited for a given set of features that have already been collected and the confidence is not sufficient. In various embodiments, a model is built or updated when a new feature suggestion is desired. When the set of not measured features is the same (e.g., Start with Vitals, and suggest measurement like CMP, CBC, specialized biomarkers), in various embodiments, the model building process may be done only once.

Step 1302 includes generating importance vectors based on features assumed to be present for all subjects in H. In various embodiments, step 1302 includes, for each subject s in the master dataset, retrieving an observation Xₛ that corresponds to one with the maximal number of features present available during a relevant timeframe. Let S refer to the set Xₛ for all s. In various embodiments, a subset of features in Xₛ either belongs to P or M. P is the set of features assumed to be present, while M is the set of features assumed to be collected after P, and there may be k features in M. In various embodiments, step 1302 may also include building a model f to predict an outcome Y using S and calculating a variance of the prediction f(S) for all s in S using standard methods (e.g standard error of prediction interval, jackknife estimators, Bayesian estimators, maximum-likelihood based estimators, and the like). The variance is an s×1 vector, V, where there is an entry in V for each s.

In various embodiments, step 1302 includes, for all subjects s in S and for j in 1 . . . k: taking the jth entry of Mₛ (which corresponds to M_{s,j}) and randomly replace it with a different value. This can be done by either picking a random value of the same feature of other subjects, or drawing from a conditional distribution modeling this feature using the remaining other features using Markov Monte Carlo based methods; using the replaced new value and pretending that it is the originally observed value in M_{s,j}, and using model to produce prediction value; repeating the above step many times independently, and calculating the variation of the prediction Vⱼ; dividing the value by the variance estimation based on X. Denote the ratio of the two as R_{s,j}=Vⱼ/Vₛ; and performing steps (I)-(III) for all j entries of Mₛ. Sort the result by R_{s,j} from the largest to the smallest. The larger R_{s,j} is, the more important the j-th feature is for subject s.

Step 1304 includes generating a model of personalized feature importance using the present features P. Specifically, build a multi-class model g (using methods such as multinomial regression, tree-based methods, neural networks, etc.) that predicts R using P, using all subjects in the historical dataset where P is available.

Step 1306 includes, for a given subject i, providing, via g(Pᵢ), a ranking of features in Mᵢ.

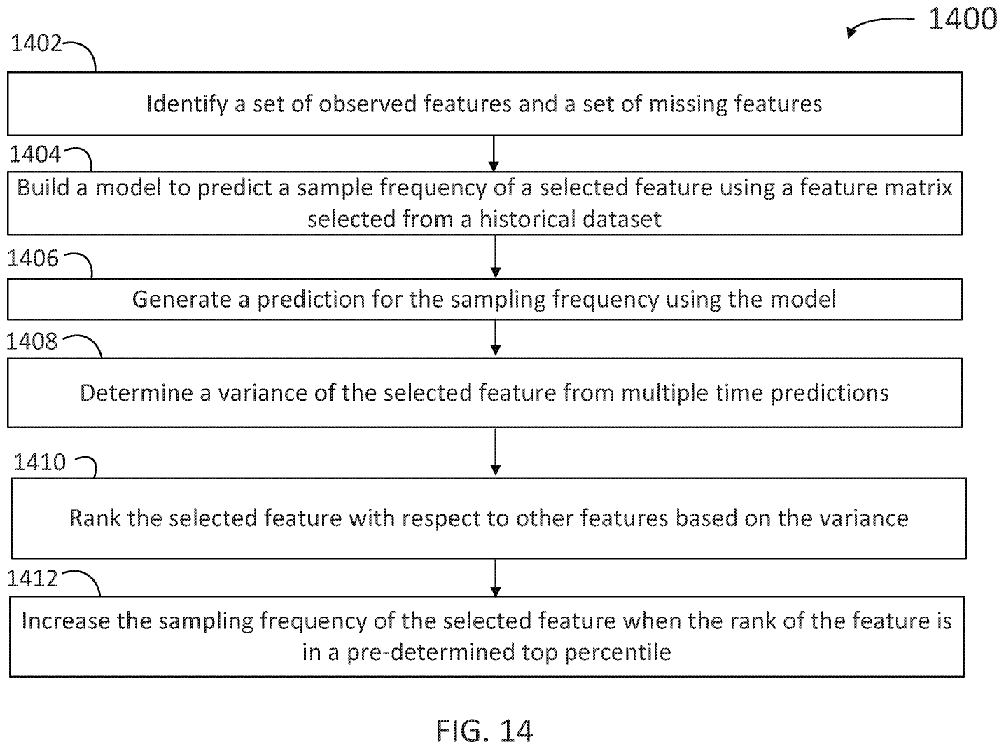

FIG. 14 is a flow chart illustrating steps in a method 1400 to determine a sampling frequency for a selected feature based on a predictability of the feature, in accordance to various embodiments. Method 1400 may be performed at least partially by any one of client devices coupled to one or more servers through a network (e.g., any one of servers 130 and any one of client devices 110, and network 150). For example, in accordance to various embodiments, the servers may host one or more medical devices or portable computer devices carried by medical or healthcare personnel. Client devices 110 may be handled by a user such as a worker or other personnel in a healthcare facility, or a paramedic in an ambulance carrying a patient to the emergency room of a healthcare facility or hospital, an ambulance, or attending to a patient at a private residence or in a public location remote to the healthcare facility. At least some of the steps in method 1400 may be performed by a computer having a processor executing commands stored in a memory of the computer (e.g., processors 212 and memories 220). In accordance to various embodiments, the user may activate an application in the client device to access, through the network, a diagnostic engine in the server (e.g., application 222 and diagnostic logic engine 240). The diagnostic engine may include a trigger logic engine, a modeling tool, a statistics tool, and an imputation tool to retrieve, supply, and process clinical data in real-time, and provide an action recommendation thereof (e.g., trigger logic engine 242, modeling tool 244, statistics tool 246, and imputation tool 248). Further, steps as disclosed in method 1400 may include retrieving, editing, and/or storing files in a database that is part of, or is communicably coupled to, the computer, using, inter-alia, the diagnostic engine (e.g., databases 252). Methods consistent with the present disclosure may include at least some, but not all, of the steps illustrated in method 1400, performed in a different sequence. Furthermore, methods consistent with the present disclosure may include at least two or more steps as in method 1400 performed overlapping in time, or almost simultaneously.

The basic idea behind this method is to estimate how predictable future values of a feature are, and based on this, determine how frequently they should be sampled. Intuitively, the less predictable the future value of a feature is, the more frequently it should be sampled. The method can formally be described as follows for a given subject i with a corresponding feature vector Pᵢ:

Step 1402 includes, for a given subject i, identifying P observed features, the missing features are M, there are j features in P, and there are k features in M. Assume we want to determine the sampling frequency, s, of a given feature, which may be either in P or M.

Step 1404 includes building a model g that predicts sₜ₊₁ using a feature matrix X. In various embodiments, step 1404 includes selecting feature matrix X from the historical dataset, H. Feature matrix X includes features exclusively in P and may include time series observations for each feature up to time t. Relevant models include autoregressive models, moving average models, markov models, and the like.

Step 1406 includes generating a prediction for sₜ₊ₓ using g(P_{0 . . . t}).

Step 1408 includes determining the variance or CV of [Pₜ,g(P_{0 . . . t})]. In various embodiments, this time-dependent variation is denoted as Vₛ. In various embodiments, the above can be extended to predicting multiple future values (e.g s_{t+x_1}, s_{t+x_2}, . . . s_{t+x_n}). In various embodiments, step 1408 includes repeating the above steps for most, or all, of the remaining features in P and M.

Step 1410 includes ranking the selected feature with respect to other features based on the variance.

Step 1412 includes increasing the sampling frequency of the selected feature when its rank is in the top r^{th} percentile. In various embodiments, step 1412 includes selecting an empirically determined factor proportional to the rank with respect to the baseline sampling frequency (as can be extracted from the historical dataset) of the feature. When the rank of the feature is in the bottom r^{th} percentile, step 1412 includes suggesting to decrease the sampling frequency by an empirically determined factor inversely proportional to the rank with respect to the baseline sampling frequency (as can be extracted from the historical dataset).

### Hardware Overview

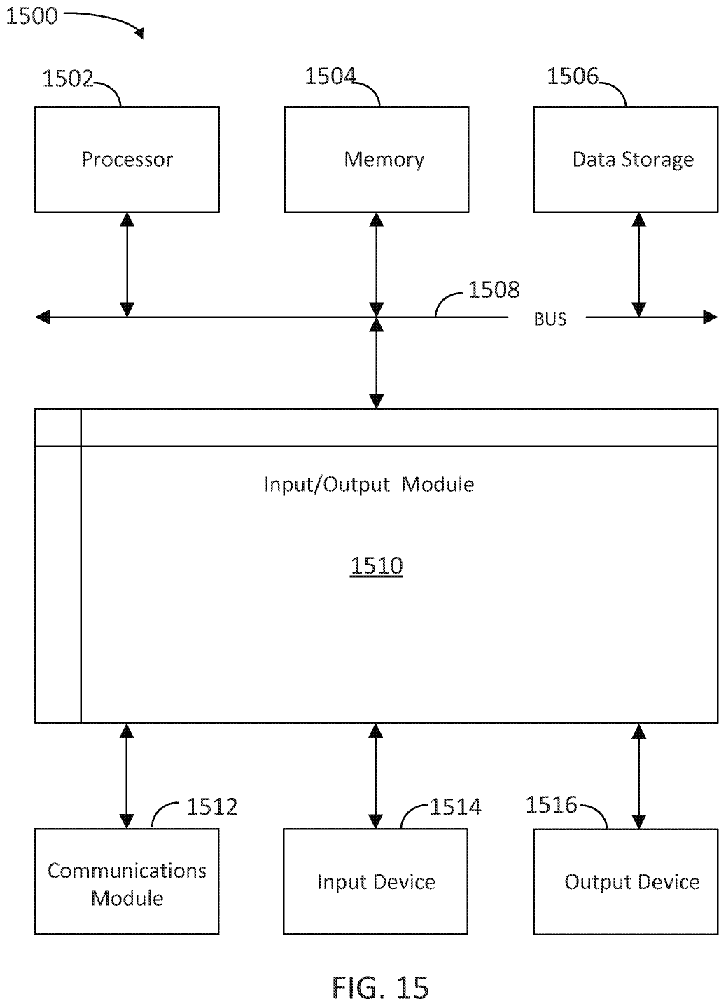

FIG. 15 is a block diagram illustrating an exemplary computer system 1500 with which the client device 110 and server 130 of FIGS. 1 and 2, and the methods of FIGS. 8 through 14 can be implemented. In certain aspects, the computer system 1500 may be implemented using hardware or a combination of software and hardware, either in a dedicated server, or integrated into another entity, or distributed across multiple entities.

Computer system 1500 (e.g., client device 110 and server 130) includes a bus 1508 or other communication mechanism for communicating information, and a processor 1502 (e.g., processors 212) coupled with bus 1508 for processing information. By way of example, the computer system 1500 may be implemented with one or more processors 1502. Processor 1502 may be a general-purpose microprocessor, a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information.

Computer system 1500 can include, in addition to hardware, code that creates an execution environment for the computer program in question, e.g., code that constitutes processor firmware, a protocol stack, a database management system, an operating system, or a combination of one or more of them stored in an included memory 1504 (e.g., memories 220), such as a Random Access Memory (RAM), a flash memory, a Read-Only Memory (ROM), a Programmable Read-Only Memory (PROM), an Erasable PROM (EPROM), registers, a hard disk, a removable disk, a CD-ROM, a DVD, or any other suitable storage device, coupled to bus 1508 for storing information and instructions to be executed by processor 1502. The processor 1502 and the memory 1504 can be supplemented by, or incorporated in, special purpose logic circuitry.

The instructions may be stored in the memory 1504 and implemented in one or more computer program products, i.e., one or more modules of computer program instructions encoded on a computer-readable medium for execution by, or to control the operation of, the computer system 1500, and according to any method well known to those of skill in the art, including, but not limited to, computer languages such as data-oriented languages (e.g., SQL, dBase), system languages (e.g., C, Objective-C, C++, Assembly), architectural languages (e.g., Java, .NET), and application languages (e.g., PHP, Ruby, Perl, Python). Instructions may also be implemented in computer languages such as array languages, aspect-oriented languages, assembly languages, authoring languages, command line interface languages, compiled languages, concurrent languages, curly-bracket languages, dataflow languages, data-structured languages, declarative languages, esoteric languages, extension languages, fourth-generation languages, functional languages, interactive mode languages, interpreted languages, iterative languages, list-based languages, little languages, logic-based languages, machine languages, macro languages, metaprogramming languages, multiparadigm languages, numerical analysis, non-English-based languages, object-oriented class-based languages, object-oriented prototype-based languages, off-side rule languages, procedural languages, reflective languages, rule-based languages, scripting languages, stack-based languages, synchronous languages, syntax handling languages, visual languages, wirth languages, and xml-based languages. Memory 1504 may also be used for storing temporary variable or other intermediate information during execution of instructions to be executed by processor 1502.

A computer program as discussed herein does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network. The processes and logic flows described in this specification can be performed by one or more programmable processors executing one or more computer programs to perform functions by operating on input data and generating output.

Computer system 1500 further includes a data storage device 1506 such as a magnetic disk or optical disk, coupled to bus 1508 for storing information and instructions. Computer system 1500 may be coupled via input/output module 1510 to various devices. Input/output module 1510 can be any input/output module. Exemplary input/output modules 1510 include data ports such as USB ports. The input/output module 1510 is configured to connect to a communications module 1512. Exemplary communications modules 1512 (e.g., communications modules 218) include networking interface cards, such as Ethernet cards and modems. In certain aspects, input/output module 1510 is configured to connect to a plurality of devices, such as an input device 1514 (e.g., input device 214) and/or an output device 1516 (e.g., output device 216). Exemplary input devices 1514 include a keyboard and a pointing device, e.g., a mouse or a trackball, by which a user can provide input to the computer system 1500. Other kinds of input devices 1514 can be used to provide for interaction with a user as well, such as a tactile input device, visual input device, audio input device, or brain-computer interface device. For example, feedback provided to the user can be any form of sensory feedback, e.g., visual feedback, auditory feedback, or tactile feedback; and input from the user can be received in any form, including acoustic, speech, tactile, or brain wave input. Exemplary output devices 1516 include display devices, such as an LCD (liquid crystal display) monitor, for displaying information to the user.

According to one aspect of the present disclosure, the client device 110 and server 130 can be implemented using a computer system 1500 in response to processor 1502 executing one or more sequences of one or more instructions contained in memory 1504. Such instructions may be read into memory 1504 from another machine-readable medium, such as data storage device 1506. Execution of the sequences of instructions contained in main memory 1504 causes processor 1502 to perform the process steps described herein. One or more processors in a multi-processing arrangement may also be employed to execute the sequences of instructions contained in memory 1504. In alternative aspects, hard-wired circuitry may be used in place of or in combination with software instructions to implement various aspects of the present disclosure. Thus, aspects of the present disclosure are not limited to any specific combination of hardware circuitry and software.

Various aspects of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. The communication network (e.g., network 150) can include, for example, any one or more of a LAN, a WAN, the Internet, and the like. Further, the communication network can include, but is not limited to, for example, any one or more of the following network topologies, including a bus network, a star network, a ring network, a mesh network, a star-bus network, tree or hierarchical network, or the like. The communications modules can be, for example, modems or Ethernet cards.

Computer system 1500 can include clients and servers. A client and server are generally remote from each other and typically interact through a communication network. The relationship of client and server arises by virtue of computer programs running on the respective computers and having a client-server relationship to each other. Computer system 1500 can be, for example, and without limitation, a desktop computer, laptop computer, or tablet computer. Computer system 1500 can also be embedded in another device, for example, and without limitation, a mobile telephone, a PDA, a mobile audio player, a Global Positioning System (GPS) receiver, a video game console, and/or a television set top box.

The term “machine-readable storage medium” or “computer-readable medium” as used herein refers to any medium or media that participates in providing instructions to processor 1502 for execution. Such a medium may take many forms, including, but not limited to, non-volatile media, volatile media, and transmission media. Non-volatile media include, for example, optical or magnetic disks, such as data storage device 1506. Volatile media include dynamic memory, such as memory 1504. Transmission media include coaxial cables, copper wire, and fiber optics, including the wires that comprise bus 1508. Common forms of machine-readable media include, for example, floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH EPROM, any other memory chip or cartridge, or any other medium from which a computer can read. The machine-readable storage medium can be a machine-readable storage device, a machine-readable storage substrate, a memory device, a composition of matter effecting a machine-readable propagated signal, or a combination of one or more of them.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

To the extent that the term “include,” “have,” or the like is used in the description or the claims, such term is intended to be inclusive in a manner similar to the term “comprise” as “comprise” is interpreted when employed as a transitional word in a claim. The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While this specification contains many specifics, these should not be construed as limitations on the scope of what may be claimed, but rather as descriptions of particular implementations of the subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

The subject matter of this specification has been described in terms of particular aspects, but other aspects can be implemented and are within the scope of the following claims. For example, while operations are depicted in the drawings in a particular order, this should not be understood as requiring that such operations be performed in the particular order shown or in sequential order, or that all illustrated operations be performed, to achieve desirable results. The actions recited in the claims can be performed in a different order and still achieve desirable results. As one example, the processes depicted in the accompanying figures do not necessarily require the particular order shown, or sequential order, to achieve desirable results. In certain circumstances, multitasking and parallel processing may be advantageous. Moreover, the separation of various system components in the aspects described above should not be understood as requiring such separation in all aspects, and it should be understood that the described program components and systems can generally be integrated together in a single software product or packaged into multiple software products. Other variations are within the scope of the following claims.

### Recitation of Embodiments

1. A method for ranking an unmeasured feature for an instance given at least one feature is measured is provided, including: imputing a first value to the unmeasured feature in the instance while holding another remaining unmeasured features constant; evaluating a first outcome with a model using the first value in the instance; imputing a second value to the unmeasured feature in the instance while holding another remaining unmeasured features constant; evaluating a second outcome with the model using the second value in the instance; determining a statistical parameter with the first outcome and the second outcome; and assigning the unmeasured feature a ranking corresponding to the statistical parameter.

2. The method of embodiment 1, further including selecting a filtered dataset from a master dataset according to at least one measured feature from the instance, the master dataset including multiple datasets associated with multiple known outcomes.

3. The method of embodiments 1 or 2, wherein assigning the unmeasured feature a ranking corresponding to the statistical parameter includes identifying, in a filtered dataset, a relative importance of the unmeasured feature with one or more known outcomes using model-based feature importance methodologies.

4. The method of any one of embodiments 1 through 3, wherein determining a statistical parameter with the first outcome and the second outcome includes accessing a master dataset including multiple datasets associated with known outcomes.

5. The method of any one of embodiments 1 through 4, wherein determining a statistical parameter with the first outcome and the second outcome includes determining a variance value associated with a model for an outcome, the model based on the unmeasured feature and at least one other distinct feature in a dataset, and evaluating a variation of prediction for an outcome with the model using multiple imputed values for the unmeasured feature in the dataset.

6. The method of any one of embodiments 1 through 5, wherein determining a statistical parameter with the first outcome and the second outcome includes: determining a rule for assessing a decision value based on a dataset, wherein the dataset includes collected values for multiple measured features in the instance and the unmeasured feature in the instance, and wherein the rule is consistent with: (1) multiple known outcomes from a master dataset that includes multiple datasets and (2) one or more measured features.

7. The method of any one of embodiments 1 through 6, wherein determining a statistical parameter with the first outcome and the second outcome includes determining an accuracy of a rule for imputing the first value to the unmeasured feature based on multiple outcome values and a known outcome for each of multiple datasets.

8. The method of any one of embodiments 1 through 7, wherein determining a statistical parameter further includes determining a time dependent variance of the first outcome and the second outcome.

9. The method of any one of embodiments 1 through 8, further including selecting a sampling frequency of the unmeasured feature based on the ranking corresponding to the statistical parameter.

10. The method of any one of embodiments 1 through 9, further including selecting a sensor device to collect a measurement from the unmeasured feature based on a precision and an accuracy of the sensor device and on the ranking of the unmeasured feature.

11. A system for ranking an unmeasured feature for an instance given at least one feature is measured is provided, the system including: a memory, storing instructions, and one or more processors communicatively coupled with the memory, and configured to execute the instructions to cause the system to: impute a first value to the unmeasured feature in the instance while holding another remaining unmeasured features constant; evaluate a first outcome with a model using the first value in the instance; impute a second value to the unmeasured feature in the instance while holding another remaining unmeasured features constant; evaluate a second outcome with the model using the second value in the instance; determine a statistical parameter with the first outcome and the second outcome; assign the unmeasured feature a ranking corresponding to the statistical parameter; and select a filtered dataset from a master dataset according to at least one measured feature from the instance, the master dataset including multiple datasets associated with multiple known outcomes.

12. The system of embodiment 11, wherein to assign the unmeasured feature a ranking corresponding to the statistical parameter the one or more processors execute instructions to identify, in a filtered dataset, a relative importance of the unmeasured feature with one or more known outcomes using model-based feature importance methodologies.

13. The system of embodiments 11 or 12, wherein to determine a statistical parameter with the first outcome and the second outcome the one or more processors execute instructions to access a master dataset including multiple datasets associated with known outcomes.

14. The system of embodiments 11 through 13, wherein to determine a statistical parameter with the first outcome and the second outcome the one or more processors execute instructions to determine a variance value associated with a model for an outcome, the model based on the unmeasured feature and at least one other distinct feature in a dataset, and evaluating a variation of prediction for an outcome with the model using multiple imputed values for the unmeasured feature in the dataset.

15. The system of embodiments 11 through 14, wherein to determine a statistical parameter with the first outcome and the second outcome the one or more processors execute instructions to determine a rule for assessing a decision value based on a dataset, wherein the dataset includes collected values for multiple measured features in the instance and the unmeasured feature in the instance, and wherein the rule is consistent with: (1) multiple known outcomes from a master dataset that includes multiple datasets and (2) one or more measured features.

16. A non-transitory, computer readable medium storing instructions which, when executed by a computer, cause the computer to perform a method for ranking an unmeasured feature for an instance given at least one feature is measured is provided, the method including: imputing a first value to the unmeasured feature in the instance while holding another remaining unmeasured features constant; evaluating a first outcome with a model using the first value in the instance; imputing a second value to the unmeasured feature in the instance while holding another remaining unmeasured features constant; evaluating a second outcome with the model using the second value in the instance; determining a statistical parameter with the first outcome and the second outcome; assigning the unmeasured feature a ranking corresponding to the statistical parameter; and selecting a filtered dataset from a master dataset according to at least one measured feature from the instance, the master dataset including multiple datasets associated with multiple known outcomes, wherein assigning the unmeasured feature a ranking corresponding to the statistical parameter includes identifying, in a filtered dataset, a relative importance of the unmeasured feature with one or more known outcomes using model-based feature importance methodologies.

17. The non-transitory, computer readable medium of embodiment 16 wherein, in the method, determining a statistical parameter with the first outcome and the second outcome includes accessing a master dataset including multiple datasets associated with known outcomes.

18. The non-transitory, computer readable medium of embodiments 16 or 17 wherein, in the method, determining a statistical parameter with the first outcome and the second outcome includes determining a variance value associated with a model for an outcome, the model based on the unmeasured feature and at least one other distinct feature in a dataset, and evaluating a variation of prediction for an outcome with the model using multiple imputed values for the unmeasured feature in the dataset.

19. The non-transitory, computer readable medium of embodiments 16 through 18 wherein, in the method, determining a statistical parameter with the first outcome and the second outcome includes determining a rule for assessing a decision value based on a dataset, wherein the dataset includes collected values for multiple measured features in the instance and the unmeasured feature in the instance, and wherein the rule is consistent with: (1) multiple known outcomes from a master dataset that includes multiple datasets and (2) one or more measured features.

20. The non-transitory, computer readable medium of embodiments 16 through 19 wherein, in the method, determining a statistical parameter with the first outcome and the second outcome includes determining an accuracy of a rule for imputing the first value to the unmeasured feature based on multiple outcome values and a known outcome for each of multiple datasets.

21. A method for ranking an unmeasured feature for an instance given at least one feature is measured, including: selecting a filtered dataset from a master dataset according to at least one measured feature from the instance, the master dataset including multiple datasets associated with multiple known outcomes; identifying, in the filtered dataset, the relative importance of the unmeasured feature with one or more known outcomes using model-based feature importance methodologies; and assigning the unmeasured feature a ranking corresponding to the output from the model-based feature importance.

22. The method of embodiment 21, wherein selecting a filtered dataset from a master dataset includes selecting at least a portion of a historical dataset.

23. The method of embodiments 21 or 22, wherein identifying, in a filtered dataset, a relative importance of the unmeasured feature with one or more known outcomes using model-based feature importance methodologies includes building or updating a model with a new feature.

24. The method of any one of embodiments 21 through 23, wherein selecting a filtered dataset further includes determining a statistical parameter with the known outcomes.

25. The method of any one of embodiments 21 through 24, wherein selecting a filtered dataset includes determining a statistical parameter with the first outcome and the second outcome includes determining a variance value associated with a model for an outcome, the model based on the unmeasured feature and at least one other distinct feature in a dataset, and evaluating a variation of prediction for an outcome with the model using multiple imputed values for the unmeasured feature in the dataset.

26. The method of any one of embodiments 21 through 25, further including determining a rule for assessing a decision value based on the filtered dataset.

27. The method of any one of embodiments 21 through 26, further including determining a statistical parameter with the multiple outcomes and determining an accuracy of a rule for imputing the first value to the unmeasured feature based on the multiple outcomes.

28. The method of any one of embodiments 21 through 27, further including determining a time dependent variance of a first outcome and a second outcome.

29. The method of any one of embodiments 21 through 28, further including selecting a sampling frequency of the unmeasured feature based on the ranking corresponding to a statistical parameter.

30. The method of any one of embodiments 21 through 29, further including selecting a sensor device to collect a measurement from the unmeasured feature based on a precision and an accuracy of the sensor device and on the ranking of the unmeasured feature.

31. A system for ranking an unmeasured feature for an instance given at least one feature is measured is provided, the system including: a memory, storing instructions; and one or more processors communicatively coupled with the memory, and configured to execute the instructions to cause the system to: select a filtered dataset from a master dataset according to at least one measured feature from the instance, the master dataset including multiple datasets associated with multiple known outcomes; identify, in the filtered dataset, the relative importance of the unmeasured feature with one or more known outcomes using model-based feature importance methodologies; and assign the unmeasured feature a ranking corresponding to the output from the model-based feature importance.

32. The system of embodiment 31, wherein to select a filtered dataset from a master dataset the one or more processors further execute instructions to select at least a portion of a historical dataset.

33. The system of embodiments 31 or 32, wherein to identify, in a filtered dataset, a relative importance of the unmeasured feature with one or more known outcomes using model-based feature importance methodologies the one or more processors execute instructions to build a model with a new feature.

34. The system of any one of embodiments 31 through 33, wherein to select a filtered dataset the one or more processors execute one or more instructions to determine a statistical parameter with the known outcomes.

35. The system of any one of embodiments 31 through 34, wherein to select a filtered dataset the one or more processors execute instructions to determine a statistical parameter with the first outcome and the second outcome and to determine a variance value associated with a model for an outcome, the model based on the unmeasured feature and at least one other distinct feature in a dataset, and to evaluate a variation of prediction for an outcome with the model using multiple imputed values for the unmeasured feature in the dataset.

36. The system of any one of embodiments 31 through 35, wherein the one or more processors further execute instructions to determine a rule for assessing a decision value based on the filtered dataset.

37. The system of any one of embodiments 31 through 36, wherein the one or more processors further execute instructions to determine a statistical parameter with the multiple outcomes and to determine an accuracy of a rule for imputing the first value to the unmeasured feature based on the multiple outcomes.

38. The system of any one of embodiments 31 through 37, wherein the one or more processors further execute instructions to determine a time dependent variance of a first outcome and a second outcome.

39. The system of any one of embodiments 31 through 38, wherein the one or more processors further execute instructions to select a sampling frequency of the unmeasured feature based on the ranking corresponding to a statistical parameter.

40. The system of any one of embodiments 31 through 39, wherein the one or more processors further execute instructions to select a sensor device to collect a measurement from the unmeasured feature based on a precision and an accuracy of the sensor device and on the ranking of the unmeasured feature.

41. A method for ranking an unmeasured feature for an instance given at least one feature is measured is provided, the method including: accessing a master dataset, the master dataset including multiple datasets associated with known outcomes; determining a variance value associated with a model for an outcome, the model based on the unmeasured feature and at least one other distinct feature in the dataset; evaluating a variation of prediction for an outcome with the model using multiple imputed values for the unmeasured feature in the dataset; and assigning the unmeasured feature a ranking according to a value of the variation of prediction relative to the variance value.

42. The method of embodiment 41, wherein accessing a master dataset includes selecting at least a portion of a historical dataset.

43. The method of embodiments 41 and 42, wherein evaluating a variation of prediction includes building or updating a model with a new feature.

44. The method of any one of embodiments 41 through 43, wherein determining a variance value associated with a model for an outcome includes selecting a filtered dataset from the master dataset.

45. The method of any one of embodiments 41 through 44, wherein determining a variance value associated with a model for an outcome, includes selecting the model based on the unmeasured feature and at least one other distinct feature in a dataset, and evaluating a variation of prediction for an outcome with the model using multiple imputed values for the unmeasured feature in the dataset.

46. The method of any one of embodiments 41 through 45, further including determining a rule for assessing a decision value based on the master dataset.

47. The method of any one of embodiments 41 through 46, further including determining an accuracy of a rule for imputing a first value to the unmeasured feature based on the known outcomes.

48. The method of any one of embodiments 41 through 47, further including determining a time dependent variance of a first outcome and a second outcome.

49. The method of any one of embodiments 41 through 48, further including selecting a sampling frequency of the unmeasured feature based on the ranking corresponding to a statistical parameter.

50. The method of any one of embodiments 41 through 49, further including selecting a sensor device to collect a measurement from the unmeasured feature based on a precision and an accuracy of the sensor device and on the ranking of the unmeasured feature.

51. A system for ranking an unmeasured feature for an instance given at least one feature is measured is provided, including: a memory, storing instructions; and one or more processors communicatively coupled with the memory, and configured to execute the instructions to cause the system to: access a master dataset, the master dataset including multiple datasets associated with known outcomes; determine a variance value associated with a model for an outcome, the model based on the unmeasured feature and at least one other distinct feature in the dataset; evaluate a variation of prediction for an outcome with the model using multiple imputed values for the unmeasured feature in the dataset; and assign the unmeasured feature a ranking according to a value of the variation of prediction relative to the variance value.

52. The system of embodiment 51, wherein to access a master dataset the one or more processors execute instructions to select at least a portion of a historical dataset.

53. The system of embodiments 51 and 52, wherein to evaluate a variation of prediction the one or more processors execute instructions to build or update a model with a new feature.

54. The system of any one of embodiments 51 through 53, wherein to determine a variance value associated with a model for an outcome the one or more processors execute instructions to select a filtered dataset from the master dataset.

55. The system of any one of embodiments 51 through 54, wherein to determine a variance value associated with a model for an outcome, the one or more processors execute instructions to select the model based on the unmeasured feature and at least one other distinct feature in a dataset, and to evaluate a variation of prediction for an outcome with the model using multiple imputed values for the unmeasured feature in the dataset.

56. The system of any one of embodiments 51 through 55, wherein the one or more processors further execute instructions to determine a rule for assessing a decision value based on the master dataset.

57. The system of any one of embodiments 51 through 56, wherein the one or more processors further execute instructions to determine an accuracy of a rule for imputing a first value to the unmeasured feature based on the known outcomes.

58. The system of any one of embodiments 51 through 57, wherein the one or more processors further execute instructions to determine a time dependent variance of a first outcome and a second outcome.

59. The system of any one of embodiments 51 through 58, wherein the one or more processors further execute instructions to select a sampling frequency of the unmeasured feature based on the ranking corresponding to a statistical parameter.

60. The system of any one of embodiments 51 through 59, wherein the one or more processors further execute instructions to select a sensor device to collect a measurement from the unmeasured feature based on a precision and an accuracy of the sensor device and on the ranking of the unmeasured feature.

61. A method for ranking an unmeasured feature for an instance given at least one feature is measured is provided, the method including: determining a rule for assessing a decision value based on a dataset, wherein the dataset includes collected values for multiple measured features in the instance and the unmeasured feature in the instance, and wherein the rule is consistent with: (1) multiple known outcomes from a master dataset that includes multiple datasets and (2) one or more measured features; determining an accuracy of the rule based on the multiple outcome values and the known outcomes for each of the datasets; and assigning the unmeasured feature a ranking corresponding the accuracy of the rule.

62. The method of embodiment 61, wherein assessing a decision value based on a dataset includes accessing a master dataset.

63. The method of embodiments 61 or 62, wherein determining an accuracy of the rule based on the multiple outcome values includes building or updating a model with a new feature.

64. The method of any one of embodiments 61 through 63, wherein determining an accuracy of the rule for assessing a decision value based on the dataset further includes determining a variance value associated with a model for an outcome.

65. The method of any one of embodiments 61 through 64, wherein determining a rule for assessing a decision value based on the dataset includes selecting a model based on the unmeasured feature and at least one other distinct feature in a dataset, and evaluating a variation of prediction for an outcome with the model using multiple imputed values for the unmeasured feature in the dataset.

66. The method of any one of embodiments 61 through 65, further including determining a rule for assessing a decision value based on a master dataset selected from an historical dataset.

67. The method of any one of embodiments 61 through 66, wherein determining an accuracy of a rule further includes updating a model for the rule with the unmeasured feature.

68. The method of any one of embodiments 61 through 67, further including determining a time dependent variance of a first outcome and a second outcome from the rule.

69. The method of any one of embodiments 61 through 68, further including selecting a sampling frequency of the unmeasured feature based on the ranking corresponding to a statistical parameter.

70. The method of any one of embodiments 61 through 69, further including selecting a sensor device to collect a measurement from the unmeasured feature based on a precision and an accuracy of the sensor device and on the ranking of the unmeasured feature.

71. A system for ranking an unmeasured feature for an instance given at least one feature is measured is provided, the system including: a memory, storing instructions; and one or more processors communicatively coupled with the memory, and configured to execute the instructions to cause the system to: determine a rule for assessing a decision value based on a dataset, wherein the dataset includes collected values for multiple measured features in the instance and the unmeasured feature in the instance, and wherein the rule is consistent with: (1) multiple known outcomes from a master dataset that includes multiple datasets and (2) one or more measured features; determine an accuracy of the rule based on the multiple outcome values and the known outcomes for each of the datasets; and assign the unmeasured feature a ranking corresponding the accuracy of the rule.

72. The system of embodiment 71, wherein to assess a decision value based on a dataset the one or more processors execute instructions to access a master dataset.

73. The system of embodiments 71 or 72, wherein to determine an accuracy of the rule based on the multiple outcome values the one or more processors execute instructions to build or update a model with a new feature.

74. The system of any one of embodiments 71 through 73, wherein to determine an accuracy of the rule for assessing a decision value based on the dataset further the one or more processors execute instructions to determine a variance value associated with a model for an outcome.

75. The system of any one of embodiments 71 through 74, wherein to determine a rule for assessing a decision value based on the dataset the one or more processors execute instructions to select a model based on the unmeasured feature and at least one other distinct feature in a dataset, and to evaluate a variation of prediction for an outcome with the model using multiple imputed values for the unmeasured feature in the dataset.

76. The system of any one of embodiments 71 through 75, wherein the one or more processors further execute instructions to determine a rule for assessing a decision value based on a master dataset selected from a historical dataset.

77. The system of any one of embodiments 71 through 76, wherein to determine an accuracy of a rule the one or more processors execute instructions to update a model for the rule with the unmeasured feature.

78. The system of any one of embodiments 71 through 77, wherein the one or more processors further execute instructions to determine a time dependent variance of a first outcome and a second outcome from the rule.

79. The system of any one of embodiments 71 through 78, wherein the one or more processors further execute instructions to select a sampling frequency of the unmeasured feature based on the ranking corresponding to a statistical parameter.

80. The system of any one of embodiments 71 through 79, wherein the one or more processors further execute instructions to select a sensor device to collect a measurement from the unmeasured feature based on a precision and an accuracy of the sensor device and on the ranking of the unmeasured feature.

81. A method to determine a sampling frequency for a selected feature based on a predictability of the feature is provided, the method including: identifying a set of observed features and a set of missing features; building a model to predict a sample frequency of a selected feature using a feature matrix selected from a historical dataset; generating a prediction for the sampling frequency using the model; determining a variance of the selected feature from multiple time predictions; ranking the selected feature with respect to other features based on the variance; and increasing the sampling frequency of the selected feature when the rank of the feature is in a pre-determined top percentile.

82. The method of embodiment 81, further including accessing a historical dataset including the observed features.

83. The method of embodiments 81 and 82, wherein building a model to predict a sample frequency includes evaluating a variation of prediction.

84. The method of any one of embodiments 81 through 83, wherein determining a variance of the selected includes selecting a filtered dataset from the master dataset.

85. The method of any one of embodiments 81 through 84, wherein determining a variance of the selected value includes selecting a model based on the observed feature and at least one other distinct feature in a dataset, and evaluating a variation of prediction for an outcome with the model using multiple imputed values for the unmeasured feature in the dataset.

86. The method of any one of embodiments 81 through 85, further including determining a rule for assessing a decision value based on the sample frequency.

87. The method of any one of embodiments 81 through 86, further including determining an accuracy of a rule for imputing a first value to the missing features based on the model.

88. The method of any one of embodiments 81 through 87, further including determining a time dependent variance of a first outcome and a second outcome.

89. The method of any one of embodiments 81 through 88, further including reducing a sampling frequency of the unmeasured feature based on a rank of the unmeasured feature.

90. The method of any one of embodiments 81 through 89, further including selecting a sensor device to collect a measurement from the unmeasured feature based on a precision and an accuracy of the sensor device and on the ranking of the unmeasured feature.

91. A system to determine a sampling frequency for a selected feature based on a predictability of the feature is provided, the system including: a memory, storing instructions; and one or more processors communicatively coupled with the memory, and configured to execute the instructions to cause the system to: identify a set of observed features and a set of missing features; build a model to predict a sample frequency of a selected feature using a feature matrix selected from a historical dataset; generate a prediction for the sampling frequency using the model; determine a variance of the selected feature from multiple time predictions; rank the selected feature with respect to other features based on the variance; and increase the sampling frequency of the selected feature when the rank of the feature is in a pre-determined top percentile.

92. The system of embodiment 91, wherein the one or more processors further execute instructions to access a historical dataset including the observed features.

93. The system of embodiments 91 or 92, wherein to build a model to predict a sample frequency the one or more processors further execute instructions to evaluate a variation of prediction.

94. The system of any one of embodiments 91 through 93, wherein to determine a variance of the selected feature the one or more processors execute instructions to select a filtered dataset from the master dataset.

95. The system of any one of embodiments 91 through 94, wherein to determine a variance of the selected value the one or more processors execute instructions to select a model based on the observed feature and at least one other distinct feature in a dataset, and to evaluate a variation of prediction for an outcome with the model using multiple imputed values for the unmeasured feature in the dataset.

96. The system of any one of embodiments 91 through 95, wherein the one or more processors further execute instructions to determine a rule for assessing a decision value based on the sample frequency.

97. The system of any one of embodiments 91 through 96, wherein the one or more processors further execute instructions to determine an accuracy of a rule for imputing a first value to the missing features based on the model.

98. The system of any one of embodiments 91 through 97, wherein the one or more processors further execute instructions to determine a time dependent variance of a first outcome and a second outcome.

99. The system of any one of embodiments 91 through 98, wherein the one or more processors further execute instructions to reduce a sampling frequency of the unmeasured feature based on a rank of the unmeasured feature.

100. The system of any one of embodiments 91 through 99, wherein the one or more processors further execute instructions to select a sensor device to collect a measurement from the unmeasured feature based on a precision and an accuracy of the sensor device and on the ranking of the unmeasured feature.

101. The method of any one of embodiments 1 through 10, wherein the other first remaining unmeasured feature is same as the other second remaining unmeasured feature.

102. The system of any one of embodiments 11 through 15, wherein the other first remaining unmeasured feature is same as the other second remaining unmeasured feature.

103. The non-transitory, computer readable medium of any one of embodiments 16-20, wherein the other first remaining unmeasured feature is same as the other second remaining unmeasured feature.

What is claimed is:

1. A method for ranking an unmeasured feature for an instance given at least one feature is measured, comprising:
imputing a first value to the unmeasured feature in the instance while holding another first remaining unmeasured feature constant;
evaluating a first outcome with a model using the first value in the instance;
imputing a second value to the unmeasured feature in the instance while holding another second remaining unmeasured feature constant;
evaluating a second outcome with the model using the second value in the instance;
determining a statistical parameter with the first outcome and the second outcome; and
assigning the unmeasured feature a ranking corresponding to the statistical parameter.

2. The method of claim 1, further comprising selecting a filtered dataset from a master dataset according to at least one measured feature from the instance, the master dataset comprising multiple datasets associated with multiple known outcomes.

3. The method of claim 1, wherein assigning the unmeasured feature a ranking corresponding to the statistical parameter comprises identifying, in a filtered dataset, a relative importance of the unmeasured feature with one or more known outcomes using model-based feature importance methodologies.

4. The method of claim 1, wherein determining a statistical parameter with the first outcome and the second outcome comprises accessing a master dataset comprising multiple datasets associated with known outcomes.

5. The method of claim 1, wherein determining a statistical parameter with the first outcome and the second outcome comprises determining a variance value associated with a model for an outcome, the model based on the unmeasured feature and at least one other distinct feature in a dataset, and evaluating a variation of prediction for an outcome with the model using multiple imputed values for the unmeasured feature in the dataset.

6. The method of claim 1, wherein determining a statistical parameter with the first outcome and the second outcome comprises:
determining a rule for assessing a decision value based on a dataset, wherein the dataset comprises collected values for multiple measured features in the instance and the unmeasured feature in the instance, and wherein the rule is consistent with: (1) multiple known outcomes from a master dataset that comprises multiple datasets and (2) one or more measured features.

7. The method of claim 1, wherein determining a statistical parameter with the first outcome and the second outcome comprises determining an accuracy of a rule for imputing the first value to the unmeasured feature based on multiple outcome values and a known outcome for each of multiple datasets.

8. The method of claim 1, wherein determining a statistical parameter further comprises determining a time dependent variance of the first outcome and the second outcome.

9. The method of claim 1, further comprising selecting a sampling frequency of the unmeasured feature based on the ranking corresponding to the statistical parameter.

10. The method of claim 1, further comprising selecting a sensor device to collect a measurement from the unmeasured feature based on a precision and an accuracy of the sensor device and on the ranking of the unmeasured feature.

11. A system for ranking an unmeasured feature for an instance given at least one feature is measured, comprising:
a memory, storing instructions; and
one or more processors communicatively coupled with the memory, and configured to execute the instructions to cause the system to:
impute a first value to the unmeasured feature in the instance while holding another first remaining unmeasured feature constant;
evaluate a first outcome with a model using the first value in the instance;
impute a second value to the unmeasured feature in the instance while holding another second remaining unmeasured feature constant;
evaluate a second outcome with the model using the second value in the instance;
determine a statistical parameter with the first outcome and the second outcome;
assign the unmeasured feature a ranking corresponding to the statistical parameter; and
select a filtered dataset from a master dataset according to at least one measured feature from the instance, the master dataset comprising multiple datasets associated with multiple known outcomes.

12. The system of claim 11, wherein to assign the unmeasured feature a ranking corresponding to the statistical parameter the one or more processors execute instructions to identify, in a filtered dataset, a relative importance of the unmeasured feature with one or more known outcomes using model-based feature importance methodologies.

13. The system of claim 11, wherein to determine a statistical parameter with the first outcome and the second outcome the one or more processors execute instructions to access a master dataset comprising multiple datasets associated with known outcomes.

14. The system of claim 11, wherein to determine a statistical parameter with the first outcome and the second outcome the one or more processors execute instructions to determine a variance value associated with a model for an outcome, the model based on the unmeasured feature and at least one other distinct feature in a dataset, and evaluating a variation of prediction for an outcome with the model using multiple imputed values for the unmeasured feature in the dataset.

15. The system of claim 11, wherein to determine a statistical parameter with the first outcome and the second outcome the one or more processors execute instructions to determine a rule for assessing a decision value based on a dataset, wherein the dataset comprises collected values for multiple measured features in the instance and the unmeasured feature in the instance, and wherein the rule is consistent with: (1) multiple known outcomes from a master dataset that comprises multiple datasets and (2) one or more measured features.

16. A non-transitory, computer readable medium storing instructions which, when executed by a computer, cause the computer to perform a method for ranking an unmeasured feature for an instance given at least one feature is measured, the method comprising:
imputing a first value to the unmeasured feature in the instance while holding another first remaining unmeasured feature constant;
evaluating a first outcome with a model using the first value in the instance;
imputing a second value to the unmeasured feature in the instance while holding another second remaining unmeasured feature constant;
evaluating a second outcome with the model using the second value in the instance;
determining a statistical parameter with the first outcome and the second outcome;
assigning the unmeasured feature a ranking corresponding to the statistical parameter; and
selecting a filtered dataset from a master dataset according to at least one measured feature from the instance, the master dataset comprising multiple datasets associated with multiple known outcomes, wherein assigning the unmeasured feature a ranking corresponding to the statistical parameter comprises identifying, in a filtered dataset, a relative importance of the unmeasured feature with one or more known outcomes using model-based feature importance methodologies.

17. The non-transitory, computer readable medium of claim 16 wherein, in the method, determining a statistical parameter with the first outcome and the second outcome comprises accessing a master dataset comprising multiple datasets associated with known outcomes.

18. The non-transitory, computer readable medium of claim 16 wherein, in the method, determining a statistical parameter with the first outcome and the second outcome comprises determining a variance value associated with a model for an outcome, the model based on the unmeasured feature and at least one other distinct feature in a dataset, and evaluating a variation of prediction for an outcome with the model using multiple imputed values for the unmeasured feature in the dataset.

19. The non-transitory, computer readable medium of claim 16 wherein, in the method, determining a statistical parameter with the first outcome and the second outcome comprises determining a rule for assessing a decision value based on a dataset, wherein the dataset comprises collected values for multiple measured features in the instance and the unmeasured feature in the instance, and wherein the rule is consistent with: (1) multiple known outcomes from a master dataset that comprises multiple datasets and (2) one or more measured features.

20. The non-transitory, computer readable medium of claim 16 wherein, in the method, determining a statistical parameter with the first outcome and the second outcome comprises determining an accuracy of a rule for imputing the first value to the unmeasured feature based on multiple outcome values and a known outcome for each of multiple datasets.

21. The method of claim 1, wherein the other first remaining unmeasured feature is same as the other second remaining unmeasured feature.

22. The system of claim 11, wherein the other first remaining unmeasured feature is same as the other second remaining unmeasured feature.

23. The non-transitory, computer readable medium of claim 16, wherein the other first remaining unmeasured feature is same as the other second remaining unmeasured feature.
